(19) United States
(12) Patent Application Publication
(10) Pub. No.: US 2010/0322878 A1
(43) Pub. Date: Dec. 23, 2010

(54) Personal Care Composition Comprising a Synthetic Cationic Polymer

(76) Inventors: Qing Stella, Cincinnati, OH (US); Edward Dewey Smith, III, Mason, OH (US); Todd Ryan Thompson, Loveland, OH (US); Beth Ann Schubert, Maineville, OH (US); Nathan Ray Whitely, Liberty, OH (US); Cynthia Ann Garza, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/817,786

(22) Filed: Jun. 17, 2010

Related U.S. Application Data
(60) Provisional application No. 61/218,342, filed on Jun. 18, 2009.

(51) Int. Cl. A61K 8/81; A61Q 19/00; A61Q 17/04; A61P 17/10; A61P 31/00; A61P 17/18

(52) U.S. Cl. 424/59; 514/772.4; 424/65; 424/642

(57) ABSTRACT

A personal care composition that comprises a synthetic random polymer comprising a net positive charge; said synthetic random polymer comprising an acrylamide monomer unit; and a cationic monomer unit comprising 3 or more positive charges; wherein said synthetic random polymer comprises a ratio of said acrylamide monomer unit to said cationic monomer unit comprising from about 55:45 to about 97:3; and a surfactant component; and an aqueous carrier.

### CROSS REFERENCE TO RELATED APPLICATION

This application claims the benefit of U.S. Provisional Application No. 61/218,342 filed Jun. 18, 2009.

### FIELD OF THE INVENTION

This invention relates to personal care compositions which comprise a random synthetic polymer with a specified ratio of monomers that enhances coacervate formation, the size and the viscoelasticity of the coacervate results in improved deposition of cosmetic agents.

### BACKGROUND OF THE INVENTION

Many commercially available personal care compositions attempt to provide skin-conditioning and cosmetic benefits; however, these do not provide sufficient deposition of cosmetic agents onto skin and hair during the application process. Without sufficient deposition from the personal care compositions, large proportions of cosmetic agents are rinsed away during the application process which adds to the overall cost of the personal cleansing composition with little benefit received from the cosmetic agents added. Conventionally, to achieve sufficient deposition a cationic polymer is added along with a limited amount of anionic surfactant to the personal care composition, so as to form adequate levels of coacervates upon dilution of the personal care composition. The formation of coacervates is important to improving deposition of cosmetic agents. While limitation of the amount of anionic surfactant may assist in deposition, it generally limits the lather performance of the personal care composition or increases the raw material cost due to the addition of more expensive surfactants to achieve good lather.

Without wishing to be bound by theory, the amount of coacervates that are formed, as well as the physicochemical characteristics of the coacervates formed are important to deposition. Size, rheological and adhesive characteristics of the coacervates formed upon dilution of a personal care composition contribute to the amount of deposition of benefit agents. It is further believed that the charge density of the cationic polymer used affects the characteristics of the coacervates formed. It is believed that certain synthetic cationic polymers possess charges randomly distributed along the length of the polymer. While these random charge characteristics of synthetic cationic polymers increase coacervate formation, they also increase the ability of the synthetic cationic polymer to interact with surfactants at multiple sites along the length of the polymer causing the formation of coacervates that are too elastic and/or non-compliant which insufficiently deposit onto the skin and hair.

Thus, there is unmet need for low cost, high lathering personal care compositions with improved cosmetic deposition that form coacervates with optimized viscoelastic properties.

### SUMMARY OF THE INVENTION

The present invention meets the aforementioned need by providing a personal care composition comprising:
a. a synthetic random polymer comprising a net positive charge; said synthetic random polymer comprising:
   i. a acrylamide monomer unit of the following formula:

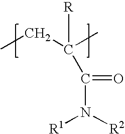

where R, R¹ and R² are a hydrogen; and
ii. a cationic monomer unit comprising 3 or more positive charges; said cationic monomer unit of the following formula:

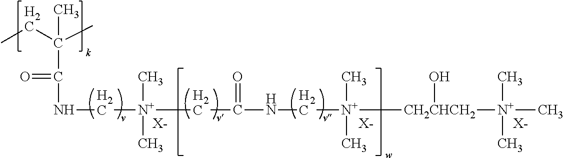

where k comprises an integer of 1, each of v, v′, and v″ independently comprise an integer from 1 to 4, w comprises an integer from 1 to 10, and X⁻ comprises a chloride anion;
wherein said synthetic random polymer comprises a molar ratio of said acrylamide monomer unit to said cationic monomer unit comprising from about 55:45 to about 97:3; and
b. an anionic surfactant component;
c. an aqueous carrier; and
d. one or more water insoluble cosmetic actives;
wherein upon dilution the personal care composition comprises one or more coacervates.

The present invention also relates to a method of treating a skin surface resulting in a modification in appearance of the skin surface using the compositions of the present invention comprising the steps of topically applying the personal care compositions of the present invention onto a skin surface and subsequently removing at least part of the personal care composition of the present invention from the skin surface within minutes; wherein the modification in the appearance of the skin surface comprises from about 1 to about 25% increase in a Delta L value as compared to a skin surface tropically treated with water.

In some embodiments, the cationic monomer of the present invention comprises a w that is equal to 1. In some embodiments, the synthetic random polymer comprises a molar ratio of the acrylamide monomer unit to the cationic monomer unit comprising about 95:5. In some embodiments, the personal care composition further comprises a hydrophobic component.

### BRIEF DESCRIPTION OF THE DRAWINGS

FIG. 1 illustrates enhanced deposition of cosmetic actives from a personal care composition comprising a random synthetic polymer comprising a triquat monomer versus the deposition from a personal care composition comprising a random synthetic polymer comprising a diquat monomer.

FIG. 2 demonstrates the importance of the molar ratio of the acrylate monomer to cationic monomer in the random synthetic polymer on deposition of cosmetic actives from a personal care composition.

FIG. 3 illustrates the effect that the elastic modulus (G′) of the coacervates formed upon dilution of a personal care composition has on deposition of cosmetic agents from a personal care composition.

FIG. 4 illustrates the effect that the size of the coacervates formed upon dilution of a personal care composition has on the deposition of cosmetic agents from a personal care composition.

FIG. 5 illustrates the effect that the addition of a hydrophobic component into the personal care composition of the present invention has on deposition of cosmetic agents from the personal care composition of the present invention.

### DETAILED DESCRIPTION OF THE INVENTION

The term “acrylamide monomer” or “acrylamide monomer unit,” as used herein refers to the chemical compound in the class of nonionic monomers, defined by one of the following structures:

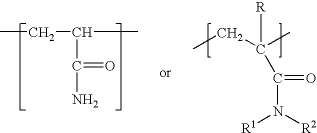

where R, R¹ and R² are a hydrogen.

The term “cationic monomer,” or “cationic monomer unit,” as used herein refers to the polyfunctional chemical compound defined by the following structure:

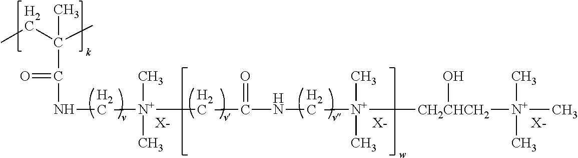

where k comprises an integer of 1, each of v, v′, and v″ independently comprises an integer from 1 to 4, w is an integer from to 1 to 10, and X⁻ is an anion. In some embodiments, each of v and v″ independently, comprise an integer of 3. In some embodiments, v′ comprises an integer of 1. In some embodiments, the w comprises an integer of from 1 to 3. In some embodiments, the w comprises an integer of 1. In some embodiments, the anion comprises a chloride anion. In some embodiments, the cationic monomer unit is a structure referred to as a “diquat,” wherein v and v″ independently comprise an integer of 2, v′ comprises an integer of 1, w comprises an integer of 0, and X⁻ comprises a chloride anion. In some embodiments, the cationic monomer unit is a structure referred to as a “triquat,” wherein v and v″ independently comprise an integer of 3, v′ comprises an integer of 1, w comprises an integer of 1, k comprises an integer of 1 and X⁻ comprises a chloride anion.

The “charge density” as used herein for the random synthetic polymer is calculated by the following equation:

C*R_{c}*1,000/(R_{c}*M_{c}+Rₙ* Mₙ)=Charge Density expressed in meq/gm

Where: C=number of cationic charges per cationic monomer; R_{c}=mole % of cationic monomer; Rₙ=mole % of nonionic monomer; M_{c}=molecular weight of the cationic monomer, excluding anionic salt (e.g., ex. Cl⁻ ions); and Mₙ=molecular weight of the nonionic monomer. An example of using the equation for to calculate the charge density of the polymer AM:Triquat substituting the following values in the above equation, where: C=3 charges; R_{c}=5%; Rₙ=95%; M_{c}=431 amu and Mₙ=71 amu

3*5%*1,000/(5%*431+95%*71)CD=1.7 meq/gm

The term “coacervate,” as used herein, refers to the physicochemical complex formed between random synthetic polymer and surfactant component within the personal care composition upon dilution of the personal care composition. Coacervate formation is dependent upon a variety of factors, such as polymer molecular weight, component concentration, ratio of components, ionic strength, charge density, the types of surfactants, the pH of the composition and the temperature of the composition. Coacervate systems and the effect of these parameters have been described, for example, in J. Caelles et al., Anionic and Cationic Compounds in Mixed Systems, 106 Cosmetics & Toiletries 49, 49-54 (April 1991), C. J. van Oss, Coacervation, Complex-Coacervation and Flocculation, 9 J. Dispersion Science and Tech., 561, 561-573, (1988-89), and in D. J. Burgess, Practical Analysis of Complex Coacervate Systems, 140 (1) J. of Colloid and Interface Science, 227, 227-238, (November 1990), all of which descriptions are incorporated herein by reference.

In some embodiments where the personal care composition does not comprise a colloidal component and the coacervate is comprised of the random synthetic polymer-surfactant component complex. The term “colloidal component” as used herein refers to a hydrophobic component, one or more water insoluble cosmetic actives and one or more colloidal suspending agents. In some embodiments where the personal care composition comprises colloidal components, the colloidal components are comprised within the physical space defined by the coacervates. In some embodiments where the personal care composition comprises colloidal components, the colloidal components are comprised on the surface of the coacervates.

Coacervates within the personal care composition of the present invention comprise a chord length of about 30 μm to about 2 mm, as measured by the Coacervate Size Measurement Methods described below. In some embodiments, one or more coacervates comprise a chord length of about 40 μm to about 1 mm. In some embodiments, one or more coacervates have a chord length of about 55 μm to about 0.5 mm. In some embodiments, one or more coacervates have a chord length of about 50 μm to about 0.1 mm.

The term “elastic modulus,” as used herein refers to the elastic modulus of the coacervates which are measured by the Coacervate Rheology Method described below, designated as G′. In some embodiments, one or more coacervates comprise an elastic modulus of about 100 Pa to about 20,000 Pa, measured by the Coacervate Rheology Method Described below. In some embodiments, one or more coacervates comprise an elastic modulus of about 200 Pa to about 10,000 Pa. In some embodiments, one or more coacervates comprise an elastic modulus of about 300 Pa to about 8,000 Pa. In some embodiments, one or more coacervates comprise an elastic modulus of about 400 Pa to about 5,000 Pa. In some embodiments, one or more coacervates comprise an elastic modulus of about 500 Pa to about 4,000 Pa.

The term “hydrophobic component,” as used herein refers to a multifunctional chemical component having a comprises a Vaughan Solubility Parameter of from about 5 (cal/cm³)^{1/2} to about 14 (cal/cm³)^{1/2} as defined by C. D. Vaughan, Solubility, Effects in Product, Package, Penetration and Preservation, 103 Cosmetics and Toiletries, 47-69 (1988). In some embodiments, the hydrophobic component comprises a Newtonian viscosity profile. A Newtonian viscosity profile means that the properties of the hydrophobic component have a linear relationship between shear stress and strain rate. A Newtonian viscosity profile means that the viscosity of the hydrophobic component is not shear rate dependent. Specifically, a Newtonian viscosity profile comprises a viscosity of 100 to 50,000 centipoise and, in some embodiments, comprises a viscosity of 1,000 to 50,000 centipoise. In some embodiments, the hydrophobic component comprises a pseudo-plastic, non-Newtonian viscosity profile, wherein the coacervate thins with increasing shear rate, and may comprise a yield stress. Specifically, a pseudoplastic, non-Newtonian viscosity profile means that the hydrophobic component has a viscosity at low shear of greater than about 3000 centipoise and in some embodiments, greater than about 7000 centipoise, and in some embodiments, greater than about 10,000 centipoise at a shear stress of about 0.05 Pascal (Pa). In some embodiments, the personal care composition of the present invention comprises at least two hydrophobic benefit agents wherein one hydrophobic component comprises a viscosity profile that is diverse from the second hydrophobic component. In some embodiments, the hydrophobic component functions as a deposition aid. In some embodiments where the hydrophobic component functions as a deposition aid, the hydrophobic component comprises a Newtonian viscosity profile and is difficult to emulsify (e.g. castor oil, mineral oils, high viscosity mineral oil and silicone oils). In some embodiments, the hydrophobic components with a non-Newtonian viscosity profile function as both a deposition aid and a cosmetic active. For example, hydrophobic components such as petrolatum, triglyceride oils, gelled mineral oil, and gelled vegetable oil, comprising crystalline and non crystalline structurants like waxes, thickeners, and polymer tend to comprise more than one function in compositions of the present invention.

The term “optical modifier,” as used herein refers to non-colored and colored, organic and inorganic materials selected from organic pigments, inorganic pigments, interference pigments, hydrophobically modified non-platelet particles, particles, platy materials, skin lightening agents, skin tanning agents, polymers and fillers. Optical modifiers, in some embodiments, include titanium oxide, zinc oxide, colored iron oxide, silicates, natural/alkaloid (including derivatives) polymers, polyethylene, alkaline earth carbonates. Platy materials, in some embodiments, comprise talc, sericite, mica, synthetic mica, barium sulfate. Particles, in some embodiments, composed of several materials like dyes, lakes, toners. The term interference pigment, as used herein refers to such as those disclosed in U.S. Pat. No. 6,395,691 (filed Feb. 28, 2001), U.S. Pat. No. 6,645,511 (filed Jan. 16, 2002), U.S. Pat. No. 6,759,376 (filed Sep. 11, 2002) and U.S. Pat. No. 6,780,826 (filed Sep. 11, 2002). Optical modifiers, in some embodiments, comprise a mixture of particles, each containing characteristics of a specific visual benefit, to create a combination of visual effects.

The term “polymer” as used herein refers to chemical compositions made by chemical or biological polymerization of monomers. A copolymer refers to a polymer comprising two monomers. A terpoplymer refers to a copolymer comprising at least 3 unique monomers.

The term “random,” as used herein, refers to the description of a polymer such that the monomer units are not arranged in a pre-determined pattern along the molecular chain where the reactivity ratio and mole percentage influence the probability of one monomer being added to the chain versus the other monomer.

The term “tottle,” as used herein, refers to a bottle that has as its base on its neck or mouth, through which its contents are filled and dispensed from. This base is also the end upon which the bottle is intended to rest or sit upon for storage by the consumer and/or for display on the store shelf. Typically, the closure on a tottle is flat or concave, such that, when the tottle is stored or displayed it rests on the closure. Suitable tottles are described in U.S. Pat. No. 7,527,077 (filed Feb. 25, 2005).

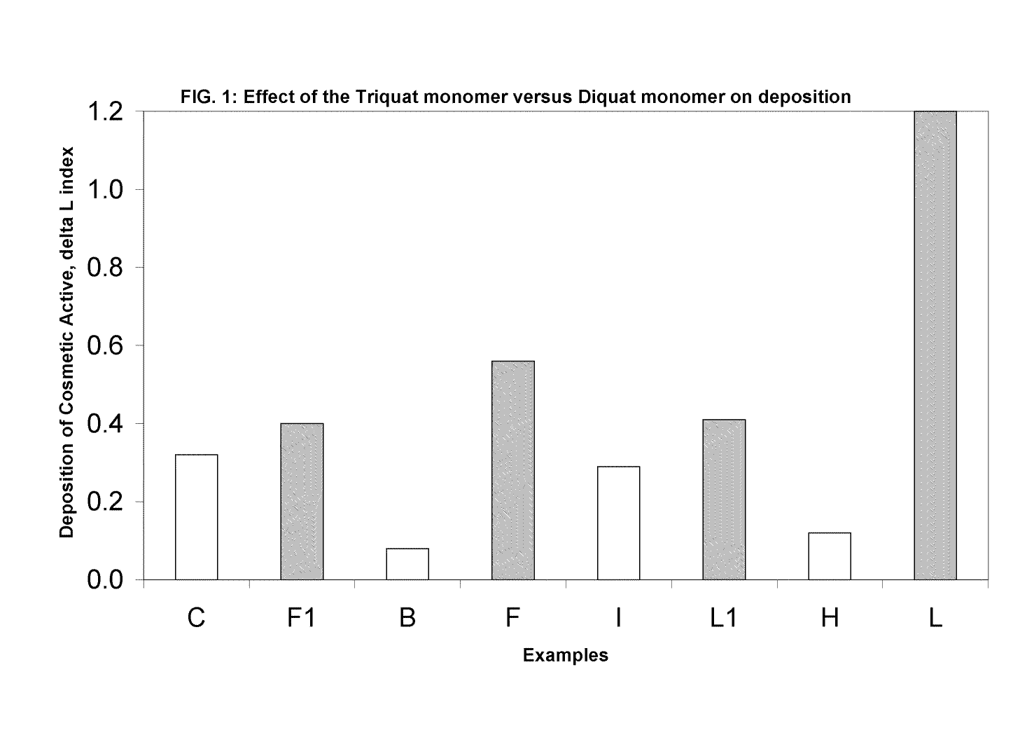

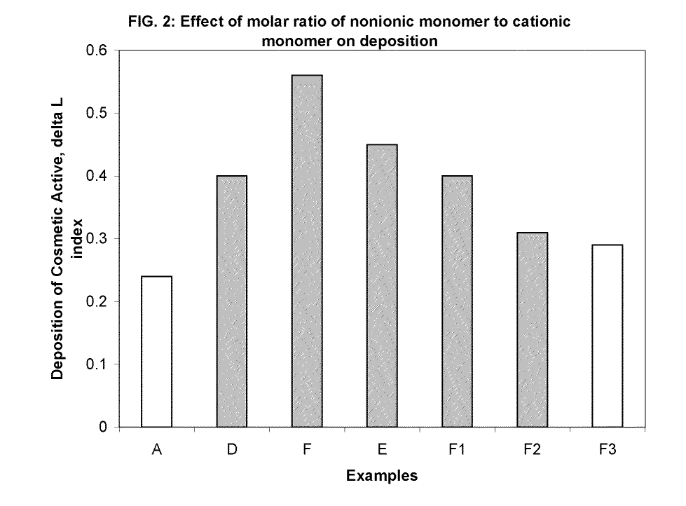

Without wishing to be bound by theory, the synthetic random polymers of the present invention that comprise a molar ratio of monomers with specified ratio of cationic monomer to nonionic monomer provide improved deposition of cosmetic agents to the skin and hair, as demonstrated in FIG. 1 and FIG. 2. These select polymers are effective at improving deposition of cosmetic agents to the skin and hair through coacervate formation upon dilution of the personal care composition at, for example, a dilution ratio between 1:0.1 and 1:50 in one embodiment. It is known that coacervates traditionally form from polymers that become insoluble and condense into distinct phases, mediated by interaction of the polymers with surfactants.

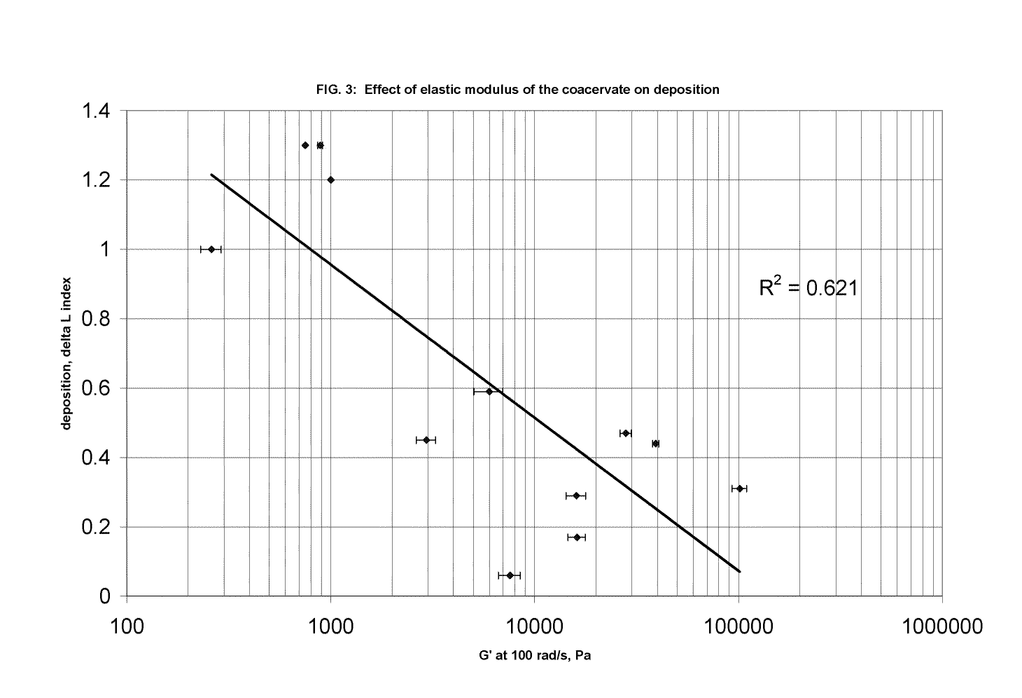

It is believed that the rheological properties of the coacervate itself formed upon dilution of a personal care composition impact the deposition of cosmetic agents. Not wishing to be bound by theory, it is believed that coacervates comprising optimal rheological properties for promoting deposition of cosmetic agents are formed in part by random synthetic polymers that possess a molar ratio of monomers and a highly localized charge density located on the cationic monomers. It has been found that some random synthetic polymers that do not possess sufficiently localized charge density tend to form an increased number of crosslinks in the resultant coacervates. The increased number of crosslinks between the polymer and the surfactant adversely affects the rheological properties of the coacervate itself, such that the coacervates are insufficiently compliant to adhere to the surface of the skin and hair. In turn, the formation of coacervates with an increased number of crosslinks also adversely affects the deposition of cosmetic actives from a personal care composition. The rheological properties of a coacervate are measured by the elastic modulus and FIG. 3 illustrates the effect of the elastic modulus of the coacervates (G′) on deposition of cosmetic actives. The deposition of cosmetic actives can be determined indirectly by change in L-color of the substrate in FIG. 3. As shown in FIG. 3, when the elastic modulus (G′) of the coacervates are below 20,000 Pa, the deposition of the cosmetic active, as indicated by the increase in L-color, from the personal care composition is optimized. The random synthetic polymers of the present invention have a localized charge density on the cationic monomer (3 or more charges per chain) that increases the strength of effective crosslinks between polymers mediated by surfactant interactions without significantly increasing the number of cros slinks The elasticity is derived from the ability of the long chains formed with the non ionic monomers to reconfigure themselves to distribute an applied stress. The optimized viscoelastic properties of the coacervates increase the adhesion of coacervate to skin resulting in enhanced deposition of cosmetic agents.

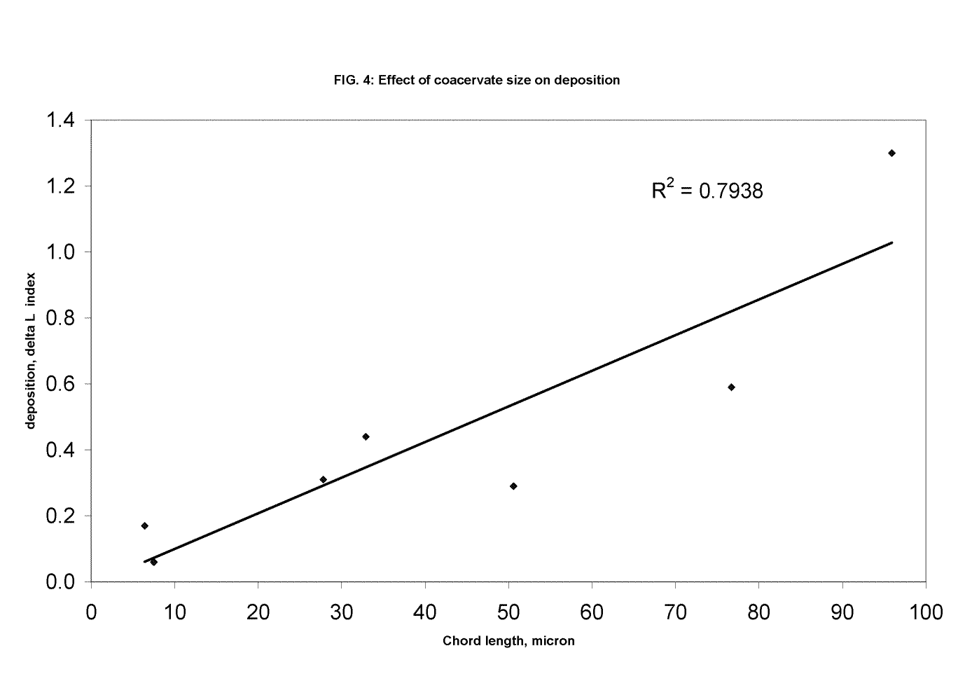

It has been found that the coacervates formed upon dilution of a personal care composition comprising a random synthetic polymer having an acrylamide monomer and cationic monomer comprising, for example, 2 or more, alternatively 3 or more positive charges form large coacervates having a chord length of from about 30 μm to about 2 mm which enhances cosmetic active deposition. FIG. 4 illustrates the effect of the size of the coacervate formed upon dilution of the personal care composition on the deposition of cosmetic agents by a personal care composition. FIG. 4 demonstrates a correlation between the size of the coacervate formed upon dilution of the personal care composition and the deposition of cosmetic actives. The trend shown in FIG. 4 is that as the size of the coacervate increases there is an increase in deposition of cosmetic active.

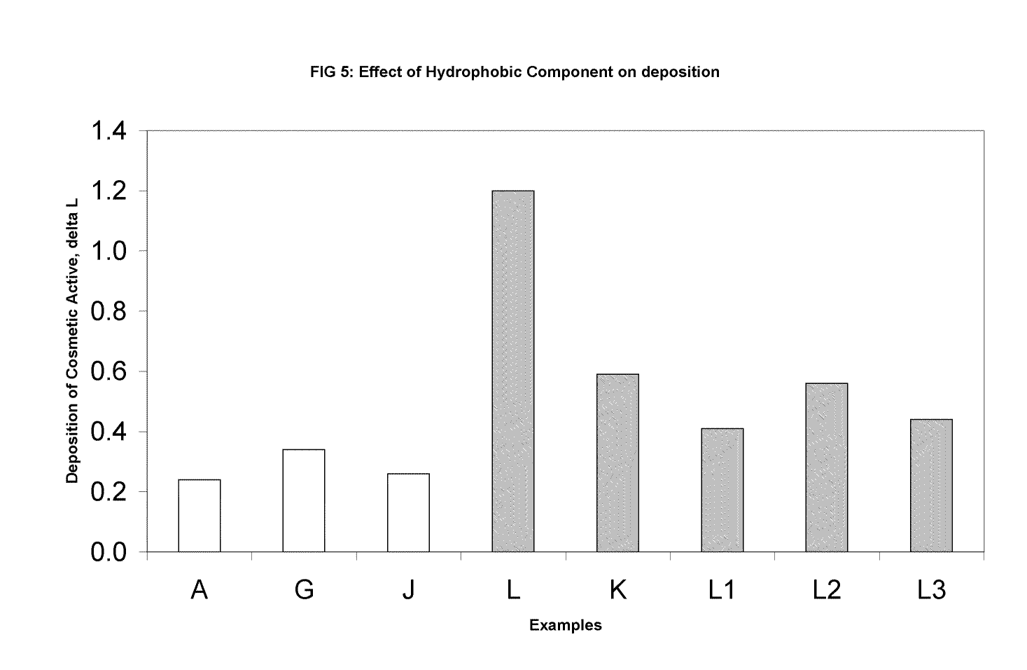

Not wishing to be bound by theory, the hydrophobic component is capable of acting as a modifier of both the rheology and the surface properties of the coacervates formed upon dilution of the personal care composition and the addition of the hydrophobic component further enhances deposition of the cosmetic active from the personal care composition of the present invention, as demonstrated in FIG. 5.

### Synthetic Random Compolymer

The personal care composition of the present invention, in some embodiments, comprises from about 0.025% to about 5.0%, by weight of personal care composition, of a synthetic random copolymer. In some embodiments, the personal care composition comprises from about 0.05% to about 3.0%, by weight of personal care composition, of a synthetic random copolymer. In some embodiments, the personal care composition comprises from about 0.1% to about 3.0%, by weight of personal care composition, of a synthetic random copolymer. In some embodiments, the personal care composition comprises from about 0.2% to about 2.0%, by weight of personal care composition, of a synthetic random copolymer. In some embodiments, the personal care composition comprises from about 0.3% to about 0.5%, by weight of personal care composition, of a synthetic random copolymer. In some embodiments, the personal care composition of the present invention comprises from about 0.2% to about 0.30%, by weight of personal care composition, of a synthetic random copolymer. In some embodiments, the personal care composition of the present invention comprises from about 0.30%, by weight of personal care composition, of a synthetic random copolymer.

The random synthetic polymer comprises a molecular weight of between about 10,000 and about 10 million in some embodiments. The random synthetic polymer comprises a molecular weight, in some embodiments, of between about 100,000 and about 3 million. In some embodiments, the random synthetic polymers comprise a charge density of about 0.1 meq/gm to about 6.8 meq/gm at the pH of intended use of the personal care composition. In some embodiments, the random synthetic polymer comprises a charge density of about 0.9 meq/gm to about 6.0 meq/gm, at the pH of intended use of the personal care composition. The pH will generally range from about pH 5 to about pH 8.

The acrylamide monomer unit comprises from about 55% to about 99.5%, by mole of the random synthetic polymer in some embodiments. In some embodiments, the acrylamide monomer unit comprises from about 70% to about 99%, by mole of the random synthetic polymer. In some embodiments, the acrylamide monomer unit comprises from about 80% to about 99%, by mole of the random synthetic polymer. In some embodiments, the acrylamide monomer unit, comprise from about 85% to about 97.5%, by mole of the random synthetic polymer.

The cationic monomer unit comprises from about 0.05% to about 45.0%, by mole of the random synthetic polymer in some embodiments. In some embodiments, the cationic monomer unit, comprises from about 1% to about 30%, by moles of the random synthetic polymer. In some embodiments, the cationic monomer unit comprises from about from about 2.5% to about 20% by moles, of the random synthetic polymer.

In some embodiments, the triquat monomer is formed by executing a three-step reaction in a jacketed reactor flask equipped with mechanical stirrer, gas inlet, condenser, and thermometer. The mechanical stirring and air purging is maintained throughout the reaction. First, 340.52 g of dimethylaminopropyl methacrylamide (DMAPMA), 238.75 g of methyl chloroacetate, 0.34 g of 4-methoxyphenol (MEHQ) and 425 g of methanol are added to the reactor and heated at about 65-70° C. for approximately 5 hours to yield (methacrylamidopropyl)(methoxy-carbonylmethyl)dimethylammonium chloride (MMDMAC). Samples are taken every 2 hours and analyzed by HPLC analysis and chloride titrated with AgNO₃ to ensure 100% conversion. Second, 0.365 g of MEHQ, and 224.5 g of dimethylaminopropylamine (DMAPA) is slowly added to MMDMAC solution after it is cooled to room temperature (about 25° C.). An exothermic reaction is observed, and the mixture appears light yellow in color. Heat is continued at about 65-70° C. for about 2 hours, then methanol is distilled out under vacuum. After confirming that all ester is converted into amide by HPLC in the second step, 637 g of 65% (3-chloro-2-hydroxypropyl)trimethylammonium chloride (Quat-188) is added. Third, the temperature is maintained at about 65-70° C. for about 2 hours. The reaction is continued in water for another hour to yield the resultant triquat monomer. The resultant triquat monomer, in some embodiments, comprises an impurity comprising multiple quats due to the excess use of chloroacetate and DMAPA. The impurities of the resultant triquat monomer do not effect polymerization and the uses of the triquat monomer. If a highly pure triquat monomer is required, the excess amount of chloroacetate and DMAPA can be removed under vacuum.

In some embodiments, the synthetic random polymer comprises a molar ratio of acrylamide monomer unit to said cationic monomer unit comprising from about 55:45 to about 99:1. In some embodiments, the synthetic random polymer comprises a molar ratio of acrylamide monomer unit to said cationic monomer unit comprising from about 60:40 to about 97.5:2.5. In some embodiments, the synthetic random polymer comprises a molar ratio of acrylamide monomer unit to said cationic monomer unit comprising from about 70:30 to about 97.5:2.5. In some embodiments, the synthetic random polymer comprises a ratio of said acrylamide monomer unit to said cationic monomer unit comprising from about 80:20 to about 95:5. In some embodiments, the synthetic random polymer comprises a ratio of said acrylamide monomer unit to said cationic monomer unit comprising from about 95:5.

The bar chart in FIG. 1 and FIG. 2 illustrates the effect of the molar ratio of monomers on deposition of cosmetic agents in a personal care composition. The personal care compositions that are gray in color in FIG. 1 and FIG. 2 correspond to some embodiments of the personal care compositions of the present invention. The personal care compositions that are white in color in FIG. 1 and FIG. 2 correspond to the comparative examples. The bar chart in FIG. 1 and FIG. 2 demonstrates greater deposition of cosmetic agents from some embodiments of the composition personal care composition versus the comparative examples and control. The ingredients of the inventive examples, comparative examples and control are shown in detail below in the Example section. FIG. 1 illustrates enhanced deposition of cosmetic actives from a personal care composition comprising a random synthetic polymer comprising a triquat monomer versus the deposition from a personal care composition comprising a random synthetic polymer comprising a diquat monomer. FIG. 2 demonstrates the importance of the molar ratio of the acrylate monomer to cationic monomer in the random synthetic polymer on deposition of cosmetic actives from a personal care composition.

### Surfactant Component

The personal care composition of the present invention comprises from about 1% to about 30%, by weight of the personal care composition, of a surfactant component. In some embodiments, the personal care composition comprises from about 3% to about 22%, by weight of the personal care composition, of a surfactant component comprising an anionic surfactant. In some embodiments, the personal care composition comprises from about 5% to 15%, by weight of the personal care composition, of the surfactant component. In some embodiments, the personal care composition comprises from about 10% to about 15%, by weight of the personal care composition, of a surfactant component. In some embodiments, the surfactant component comprises a mixture of surfactants selected from anionic surfactants, amphoteric surfactants, zwitterionic surfactants, cationic surfactants, nonionic surfactants and mixtures thereof. Suitable surfactants for the personal care composition are described in McCutcheon's: Detergents and Emulsifiers North American Edition (Allured Publishing Corporation 1947) (1986), McCutcheon's, Functional Materials North American Edition (Allured Publishing Corporation 1973) (1992) and U.S. Pat. No. 3,929,678 (filed Aug. 1, 1974).

In some embodiments, the surfactant component is an isotropic composition. In some embodiments, the surfactant component is structured such that the resultant personal care composition is a lamellar composition, or is at least partly present in the lamellar phase, including planar as well as vesicles (e.g., multilamellar vesicles).

In some embodiments, surfactant component comprises from about 1% to about 95%, by weight of the personal care composition, of at least one anionic surfactant. In some embodiments, the surfactant component comprises from about 50% to about 95%, by weight of the personal care composition, of at least one anionic surfactant. In some embodiments, at least one of the anionic surfactants comprises an ethoxylate group. In some embodiments, at least one of the anionic surfactants comprises a propoxylate group or a methoxylate group. In some embodiments, the anionic surfactant comprises alkyl sulfates and alkyl ether sulfates that have the respective formula ROSO₃M and RO(C₂H₄O)ₓSO₃M, wherein R is alkyl or alkenyl of from about 8 to about 18 carbon atoms, x is an integer having a value of from 1 to 10, and M is a cation such as ammonium, sodium, potassium, magnesium and calcium. Alkyl sulfates and alkyl ether sulfates, in some embodiments, comprise from about 10 to about 16 carbon atoms; preferably, 12 to about 14 carbon atoms. The monohydric alcohols used to make alkyl ether sulfates, in some embodiments, are synthetic; alternatively, monohydric alcohols are derived from fats (e.g. coconut oil, palm kernel oil, tallow). These monohydric alcohols, in some embodiments are reacted with molar proportions of ethylene alcohol such that the resultant mixture of molecular species has an average of 0.5 to about 3 moles ethylene oxide per mole of alcohol that is sulfated and neutralized. The hydrocarbons can be linear, branched, cyclic or mixed.

In some embodiments, the anionic surfactant comprises the water-soluble salts of organic, sulfuric acid reaction products conforming to the formula [R¹—SO₃—M] where R¹ is a straight or branched chain, saturated, aliphatic hydrocarbon radical having from about 8 to about 24 carbon atoms and M is a cation such as ammonium, sodium, potassium, magnesium and calcium. The aliphatic hydrocarbon radical, in some embodiments, comprises from about 10 to about 18 carbon atoms.

The anionic surfactants, in some embodiments, comprise reaction products of fatty acids esterified with isethionic acid and neutralized with sodium hydroxide. In some embodiments, the surfactant component comprises from about 0.1% to about 50%; alternatively, from about 0.5% to about 10%, by weight of the personal care composition, of amphoteric or zwitterionic surfactants. Suitable amphoteric or zwitterionic or amphoteric surfactants, in some embodiments, comprise those described in U.S. Pat. No. 5,104,646 (filed Jul. 16, 1990) and U.S. Pat. No. 5,106,609 (Jul. 16, 1990). The amphoteric surfactants, in some embodiments, comprise those surfactants broadly described as comprising aliphatic groups and secondary or tertiary amines in which the aliphatic moieties can be straight or branched chain and wherein one of the aliphatic substituent contain from about 8 to about 18 carbon atoms and one contains an anionic water solubilizing group such as carboxy, sulfonate, sulfate, phosphate, or phosphonate. The zwitterionic surfactants, in some embodiments, comprise those surfactants broadly described as comprising aliphatic groups and quaternary ammonium, phosphonium, and sulfonium compounds, in which the aliphatic groups can be straight or branched chain, and wherein one of the aliphatic substituents contains from about 8 to about 18 carbon atoms and contains an anionic group such as carboxy, sulfonate, sulfate, phosphate or phosphonate. In some embodiments, the zwitterionic surfactant comprises a betaine.

In some embodiments, the amphoteric or zwitterionic surfactant is selected from cocoamidopropyl betaine, lauramidopropyl betaine, coco betaine, lauryl betaine, cocoamphoacetate, cocoamphodiacetate, lauroamphoacetate, lauroamphodiacetate, lauramine oxide, sarcosinate, glutamate, lactate and mixtures thereof. The surfactant component, in some embodiments, comprise cationic surfactants that comprise amino or quaternary ammonium hydrophilic moieties which are positively charged when dissolved in the personal care composition of the present invention. Cationic surfactants are disclosed in Schwartz, et al., Surface Active Agents, Their Chemistry and Technology (Interscience Publishers) (1949); U.S. Pat. No. 3,155,591 (filed Dec. 6, 1961); U.S. Pat. No. 3,929,678 (filed Aug. 1, 1974); U.S. Pat. No. 3,959,461 (filed May 28, 1974) and U.S. Pat. No. 4,387,090 (filed Feb. 13, 1981).

In some embodiments, the surfactant component comprises non-ionic surfactants selected from cocoamide monoethanolamine, lauramide monoethanolamine, cocoyl glucosides, lauryl glucosides, decyl glucosides, other alkyl glucosides, trideceth-1, trideceth-3 from EXXAL® 23 and laureth 1, -2, -3, -4 and -5, alkyl ethoxylates from linear, branched, and unsaturated hydrocarbons.

In some embodiments, the surfactant component comprises the surfactants, selected from ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, cocamidopropyl betaine, sodium lauroamphoacetate, alkyl glyceryl ether sulfonate, and mixtures thereof.

### Cosmetic Actives

In some embodiments, the personal care compositions comprise from about 0.001% to less than about 20%; alternatively, less than about 15%; alternatively, less than about 10%; alternatively, less than about 6%; alternatively, less than about 5%; alternatively, less than about 4%; alternatively, less than about 3%; alternatively, less than about 2%; alternatively, less than about 1%; alternatively, less than about 0.5%; alternatively, less than about 0.25%; alternatively, less than about 0.1%; alternatively, less than about 0.01%, less than about 0.005%, less than about 0.001% by weight of the solid personal care composition, of one or more water insoluble cosmetic actives. One or more water insoluble cosmetic actives are selected from optical modifiers; antimicrobials (ZPT); fragrances or perfumes; deodorant actives; vitamins (e.g. Retinol); vitamin derivatives (e.g. panthenol); sunscreens, desquamation actives, such as those described in U.S. Pat. No. 5,681,852 (filed Jun. 7, 1995) and U.S. Pat. No. 5,652,228 (filed Nov. 12, 1993); zinc carbonate; anti-wrinkle actives; anti-atrophy actives (e.g. N-acetyl derivatives, thiols, phenol); anti-oxidants (e.g. ascorbic acid derivatives, tocophenol); skin soothing agents; skin healing agents (e.g. panthenoic acid derivatives, aloe vera); anti-acne medicaments; medicaments; essential oils (e.g. lavender, tea tree, violet balsam); sensates (e.g. menthol); clays (e.g. zeolites, kaolin, bentonite); and mixtures thereof. Other suitable optional ingredients are those approved for use in cosmetics described in the CTFA Cosmetic Ingredient Handbook, Second Edition (The Cosmetic, Toiletries, and Fragrance Association, Inc. 1988) (1992).

For example, the cosmetic actives can comprise one or more perfumes or perfume raw materials. According to example embodiments, the perfumes or perfume raw materials can be selected from one or more of the following: acetophenone; allylamyl_glycolate; alpha-pinene; amyl butyrate; anisic aldehyde; benzyl acetate; beta-naphthol methyl ether; citronellol; citronellyl nitrile; clonal; delta damascone; delta muscenone; ethylmethylphenylglycidate; ethyl safranate; exaltolide; fenchyl alcohol; florhydral; geraniol; helvetolide; hivernal_isomer-1; hivernal isomer-2; hydroxycitronellal; beta-ionone; laevo-carvone; linalool; linalyl isobutyrate; maltol; methyl beta-naphthyl_ketone; methyl salicylate; octylaldehyde; pt-bucinal; p-cresyl_methyl ether; para-hydroxy phenyl butanone; phenylethyl dimethyl carbinol; pomarose; terpinyl acetate; or any other suitable fragrance or perfume or mixtures thereof

### Aqueous Carrier

The personal care compositions of the present invention comprise from about 30% to about 95%, by weight of the personal care composition of an aqueous carrier. In some embodiments, the personal care composition comprises from about 60% to about 90%, by weight of the personal care composition, of an aqueous carrier. In some embodiments, the personal care composition comprises from about 75% to about 85%, by weight of the personal care composition, of an aqueous carrier. Useful aqueous carriers comprise water and water solutions of lower alkyl alcohols. In some embodiments the water solutions of lower alkyl alcohols comprise are monohydric alcohols comprising 1 to 6 carbons. The water solutions of lower alkyl alcohols comprise, in some embodiments, comprise ethanol and isopropanol. As recognized by one of ordinary skill in the art, the amount and type of aqueous carrier is selected according to the compatibility with the other ingredients of the personal care composition and the desired characteristics of the resultant personal care composition.

### Hydrophobic Component

In some embodiments, the personal care composition comprises from about from about 0.01% to about 10.0%, by weight of the personal care composition, of a hydrophobic component. In some embodiments, the personal care composition comprises from about from about 0.01% to about 5.0%, by weight of the personal care composition, of a hydrophobic component. In some embodiments, the personal care composition comprises from about 0.05% to about 2.0%; alternatively, from about 0.75% to about 1.0%, by weight of the personal care composition, of hydrophobic component. In the presence of a hydrophobic component, the personal care composition form coacervates which may comprise a hydrophobic component. Not wishing to be bound by theory, the hydrophobic component acts as a modifier of both the rheology and the surface properties of the coacervates formed upon dilution of the personal care composition. Large coacervates with optimal rheology and surface energy have an exceptional ability to deposit cosmetic actives. In some embodiments, the hydrophobic component is pre-mixed with one or more cosmetic actives prior to addition to the personal care composition. In some embodiments, the hydrophobic component is added separately to the personal care composition.

Not wishing to be bound by theory, the hydrophobic component acts as a modifier of both the rheology and the surface properties of the coacervates formed upon dilution of the personal care composition and the addition of the hydrophobic component further enhances deposition of the cosmetic active from the personal care composition of the present invention, as demonstrated in FIG. 5. The bar chart in FIG. 5 illustrates the effect of the monomer ratio on deposition of cosmetic agents in a personal care composition that further comprises a hydrophobic component. The personal care compositions that are gray in color in FIG. 5 correspond to some embodiments of the personal care compositions of the present invention. The personal care compositions that are white in color in FIG. 5 correspond to the comparative examples. The bar charts in FIG. 5 demonstrate greater deposition of cosmetic agents from some embodiments of the composition personal care composition versus the comparative examples and control. The ingredients of the inventive examples, comparative examples and control are shown in detail below in the Example section.

In some embodiments, the hydrophobic component is a water-dispersible, non-volatile liquid. Non-limiting examples of hydrophobic benefit materials having VSP values ranging from about 5 to about 14 include the following: Cyclomethicone 5.9, Squalene 6.0, Petrolatum 7.3, Isopropyl Palmitate 7.8, Isopropyl Myristate 8.0, Castor Oil 8.9, Cholesterol 9.6, Butylene Glycol 13.2, as reported in C. D. Vaughan, Solubility, Effects in Product, Package, Penetration and Preservation, 103 Cosmetics and Toiletries, 47-69 (1988).

In some embodiments, the hydrophobic component comprises hydrocarbon oils, polyolefins, fatty esters, fatty alcohols, sucrose esters, silicone oils and mixtures thereof.

In some embodiments, the hydrophobic component comprises hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Both straight and branched chain hydrocarbon oils, in some embodiments, comprise from about 12 to 19 carbon atoms.

In some embodiments, the hydrophobic component comprises hydrocarbon oils that comprise paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. In some embodiments, the hydrophobic component can comprise branched-chain isomers of hydrocarbon oils. In some embodiments, the hydrophobic component comprises polybutene that is copolymer of isobutylene and butene, which is commercially available as L-14 polybutene from Amoco Chemical Corporation.

In some embodiments, the hydrophobic component comprises liquid polyolefins, liquid poly-α-olefins and hydrogenated liquid poly-α-olefins. In some embodiments, the hydrophobic component comprises fatty esters having at least 10 carbon atoms. The fatty esters, in some embodiments, comprise hydrocarbyl chains derived from fatty acids or alcohols. The fatty esters, in some embodiments, comprise glycerides including, but not limited to, mono-glycerides, di-glycerides, and tri-glycerides. Glycerides, in some embodiments, comprise fats and oils derived from vegetables and animals. The fatty esters, in some embodiments, comprise castor oil, safflower oil, jojoba oil, cottonseed oil, corn oil, olive oil, cod liver oil, almond oil, avocado oil, palm oil, sesame oil, lanolin and soybean oil. Glycerides, in some embodiments, comprise synthetic oils including, but are not limited to, triolein, tristearin and glyceryl trilaurate.

In some embodiments, the hydrophobic component comprises fatty alcohols having at least about 10 carbon atoms. In some embodiments, organic conditioning oils comprise fatty alcohols that comprise from about 10 to about 22 carbon atoms. In some embodiments, organic conditioning oils comprise fatty alcohols that comprise from about, most preferably about 12 to about 16 carbon atoms. In some embodiments, the hydrophobic component comprises alkoxylated fatty alcohols which conform to the general formula: CH₃(CH₂)ₙCH₂(OCH₂CH₂)ₚOH wherein n is a positive integer having a value from about 8 to about 20, alternatively, from about 10 to about 14, and p is a positive integer having a value from about 1 to about 30, alternatively, from about 2 to about 5.

In some embodiments, the hydrophobic component comprises liquid sucrose esters. Liquid sucrose esters, in some embodiments, are prepared by an esterification reaction between fatty acid alkyl esters and sucrose in the presence of a catalyst (Feuge, R. O., et al., 47 J. Amer. Oil Chem. Soc. 56-60 (1970)) and in the presence or absence of a solvent (Rizzi, G. P., and Taylor, H. M., 55 J. Amer. Oil Chem. Soc. 398-401 (1978)).

The hydrophobic component, in some embodiments, comprises silicone oils selected from siloxanes, organo-modified silicones and fluoro-modified silicones. The organo-modified silicones, in some embodiments, comprise an organo group selected from alkyl groups, alkenyl groups, hydroxyl groups, amine groups, quaternary groups, carboxyl groups, fatty acid groups, ether groups, ester groups, mercapto groups, sulfate groups, sulfonate groups, phosphate groups, propylene oxide groups, and ethylene oxide groups. In some embodiments, the silicone oil is dimethicone. Suitable silicones, in some embodiments are those described in U.S. Pat. No. 2,826,551 (filed Jan. 4, 1954); U.S. Pat. No. 3,964,500 (Jun. 18, 1975) and U.S. Pat. No. 4,364,837 (filed Dec. 21, 1982). The silicones can be made by the methods disclosed in 15 Encyclopedia of Polymer Science and Engineering, 204-308 (John Wiley & Sons, Inc. 2^{nd} ed. 1989).

In some embodiments, the hydrophobic components selected from petrolatum, natural and synthetic waxes (e.g. micro-crystalline waxes, paraffins, ozokerite, lanolin wax, polyethylene, pentahydrosqualene) and mixtures thereof.

In some embodiments, the hydrophobic component comprises one or more hydrophobic components selected from castor oil, mineral oil, polybutene, jojoba oil, silicone oils, petrolatum, triglyceride oils, gelled mineral oils, gelled vegetable oils, oils comprising crystalline structurants, oils comprising non-crystalline structurants and mixtures thereof.

### Colloidal Suspending Agent

The personal care composition, in some embodiments, comprises from about 0.1% to about 10%, by weight of the personal care composition, of one or more colloidal suspending agents. The personal care composition, in some embodiments, comprise from about from about 0.3% to about 5.0%, by weight of the composition, of one or more colloidal suspending agents.

The colloidal suspending agent, in some embodiments, comprises the group selected from acyl derivatives, long chain amine oxides and mixtures thereof which are described in U.S. Pat. No. 4,741,855 (filed Jul. 21, 1987). The colloidal suspending agents, in some embodiments, comprise ethylene glycol esters of fatty acids comprising having from about 16 to about 22 carbon atoms. The colloidal suspending agents, in some embodiments, comprise ethylene glycol stearates, both mono and distearate. The ethylene glycol distearate, in some embodiments, comprises less than about 7% of the mono stearate. The colloidal suspending agents, in some embodiments, comprise alkanol amides of fatty acids comprising from about 16 to about 22 carbon atoms. The colloidal suspending agents, in some embodiments, comprise alkanol amides of fatty acids comprising from about 16 to about 18 carbon atoms. The colloidal suspending agents, in some embodiments, comprise stearic monoethanolamide, stearic diethanolamide, stearic monoisopropanolamide and stearic monoethanolamide stearate. The colloidal suspending agents, in some embodiments, comprise long chain acyl derivatives comprising long chain esters of long chain fatty acids (e.g., stearyl stearate, cetyl palmitate, etc.); glyceryl esters (e. g., glyceryl distearate) and long chain esters of long chain alkanol amides (e.g., stearamide diethanolamide distearate, stearamide monoethanolamide stearate). The colloidal suspending agents, in some embodiments, comprise long chain acyl derivatives, ethylene glycol esters of long chain carboxylic acids, long chain amine oxides, and alkanol amides of long chain carboxylic acids. In some embodiments, the colloidal suspending agents comprise long chain hydrocarbyls having C₈-C ₂₂ chains. The colloidal suspending agents, in some embodiments, comprise long chain acyl derivatives comprising N,N-dihydrocarbyl amido benzoic acid and soluble salts thereof. In some embodiments, the N,N-dihydrocarbyl amido benzoic acid and soluble salts thereof comprise N,N-di(hydrogenated) C₁₆, C₁₈ and tallow amido benzoic acid species, which are commercially available from Stepan Company (Northfield, Ill., USA).

The colloidal suspending agents, in some embodiments, comprise primary amines having a fatty alkyl moiety having at least about 16 carbon atoms. In some embodiments, primary amines having a fatty alkyl moiety having at least about 16 carbon atoms comprise palmitamine or stearamine. The colloidal suspending agents, in some embodiments, comprise secondary amines having two fatty alkyl moieties each having at least about 12 carbon atoms. In some embodiments, secondary amines having two fatty alkyl moieties each having at least about 12 carbon atoms comprise dipalmitoylamine or di(hydrogenated tallow)amine. The colloidal suspending agents, in some embodiments, comprise di(hydrogenated tallow)phthalic acid amide, crosslinked maleic anhydride-methyl vinyl ether copolymer and trihydroxystearin.

The colloidal suspending agents, in some embodiments, comprise the group selected from stearic monoethanolamide, stearic diethanolamide, stearic monoisopropanolamide, stearic monoethanolamide stearate, stearyl stearate, cetyl palmitate, glyceryl distearate, stearamide DEA distearate, stearamide MEA stearate, ethylene glycol distearate, trihydroxystearin, hydrogenated castor oil and mixtures thereof.

In some embodiments, the colloidal suspending agent comprises microfibrous cellulose. In some embodiments, the microfibrous cellulose comprises a fiber diameter of 0.1 micrometer. Suitable commercially available microfibrous cellulose is AXCEL CG-PX available from CP KELCO.

### Optional Ingredients

In some embodiments, the personal care compositions comprise from about 0.001% to less than about 20%, less than about 15%, less than about 10% less than about 6%, less than about 5%, less than about 4%, less than about 3%, less than about 2%, less than about 1%, less than about 0.5%, less than about 0.25%, less than about 0.1%, less than about 0.01%, less than about 0.005%, by weight of the solid personal care composition, of one or more optional ingredients. One or more optional ingredient are selected from electrolytes; brighteners; thickening agents (e.g. cholesterolic ingredients, dibenzylidene alditols, lanolinolic ingredients, fatty alcohols, triglycerides); preservatives; pH buffering agents; calcium carbonate; talc; baking soda; baking soda related ingredients; fungicides; bactericides; malodor absorbing ingredients; chelators, such as those described in U.S. Pat. No. 5,487,884 (filed Oct. 22, 1982); sequestrants and suitable optional ingredients are those approved for use in cosmetics described in the CTFA Cosmetic Ingredient Handbook, Second Edition (The Cosmetic, Toiletries, and Fragrance Association, Inc. 1988) (1992).

### Method of Treatment of the Skin Surface

The present invention also relates to a method of treating a skin surface resulting in a modification in appearance of the skin surface using the compositions of the present invention. The method comprises the step of topically applying the compositions of the present invention onto a skin surface. The method comprises the step of subsequently removing the personal care composition of the present invention from the skin surface. The modification in the appearance of the skin surface comprises from about 1 to about 25% increase in a Delta L value as compared to a skin surface topically treated with water. In some embodiments, the personal care composition from the skin is removed by rinsing the skin surface, wiping the skin surface with a substrate, removing the personal care composition by a device and mixtures thereof. In some embodiments, the personal care composition from the skin is removed by a device selected from a razor, electric skin brush and mixtures thereof. It is understood that when removing the personal care composition from the skin, an amount of the personal care composition optionally remains deposited on the skin For example, when rinsing the personal care composition from the surface of the skin, a effective amount remains on the skin In some embodiments, the personal care composition is removed within about 1 to about 10 minutes. In some embodiments, the modification of the skin surface is a opacity modification, a color modification, a reflectance modification. The increase in Delta L can be measured any method disclosed below comparing a skin surface that has been topically treated with water.

### Test Methods

Coacervate Isolation Method: Dilutions of personal care composition are prepared to measure coacervate, by adding the personal care composition into a clean 50 mL conical transparent centrifuge tube (the weight of which is recorded as the empty tube tare weight) followed by deionized water to achieve the desired dilution ratio by weight. 50 gm total weight of composition and water are added to the centrifuge tube. For example, 25.0 gm of personal care composition and 25.0 gm water are added for a 1:1 dilution ratio; 14.29 gm of personal care composition and 35.71 gm water are added for a 1:2.5 dilution; and so on. The centrifuge tube is placed on a tube rotator (e.g. CEL-GRO™ Tissue culture rotator) set at medium rotation speed and left to mix overnight. The centrifuge tube is centrifuged at 4500 rpm for 30 minutes at ambient temperature, so that the coacervate settles to the bottom of the centrifuge tube. The supernatant overlaying the coacervate at the top of the centrifuge tube is decanted without pouring any coacervate from the tube. (If the coacervate is fluid, decanting may comprise pipetting or other means to absolve the supernatant), discarded and excess supernatant is dried from the interior walls of the centrifuge tube without touching the coacervate. The centrifuge tube is weighed to determine weight of the coacervate by subtracting the empty tube tare weight. Amount of coacervate is reported at dilution ratios between, for example, 1:0.1 and 1:50 including 1:0.1, 1:1, 1:2.5, 1:5, 1:9, 1:50 as % coacervate according to the following equation:

% coacervate=(weight of coacervate/weight of personal care composition added to make dilution)×100%

In some embodiments where the personal care composition, does not comprise a colloidal component (e.g. hydrophobic component, cosmetic actives, colloidal suspending agents) the coacervates primarily comprises polymer-surfactant complex. In some embodiments where the personal care composition colloidal components, the colloidal components may be comprised within the coacervates.

Coacervate Size Measurement Method: The size and structure of the coacervates can be measured both in the neat and dilute personal care composition.

Coacervate size measurement in a neat composition: The size and structure of the coacervates can be measured in the neat personal care composition, if it forms with no dilution, by light microscopy. A small drop of personal care composition is placed on a glass microscope slide and covered with a glass coverslip. The coacervates are identified by their birefringence indicating a liquid colloidal character either by comparison of the properties of the personal care composition in the absence of cationic polymer or by systematic comparison of other components in the personal care composition. Image analysis of microscopy pictures of the coacervates with the personal care composition are used to quantify the size of the coacervates. In some embodiments, enhanced contrast techniques are used to improve contrast between the coacervates and the surrounding liquid, including differential interference contrast, phase contrast, polarized light, and/or the use of fluorescent dyes. Additional samples of the personal care composition are imaged to ensure that the resulting images and coacervate sizes are representative of the entire personal care composition.

Coacervate size measurement in a diluted composition: Measurement of the coacervate size in a diluted composition requires sample preparation using a bench-top dilution method. Dilution ratios such as 1:0.1, 1:1, 1:2.5, 1:5, 1:9, or 1:50 are measured. For a 1:1 dilution, 1000 gm deionized water at 20-25° C. is placed in a 3 liter stainless steel beaker and stirred using a standard laboratory mixer with impeller blade set at 500 rpm to create a small vortex. 1000 gm personal care composition is added into the water maintaining sufficient agitation to mix without creating air bubbles.

Measurement of the coacervate size in a diluted composition is determined by laser scattering, preferably focused beam reflectance measurement. The laser scattering techniques comprise laser diffraction with Mie theory, dynamic light scattering, focused beam reflectance mode and mixtures thereof. The choice of scattering method depends on the coacervate size and the concentration level of coacervates in solution. Dynamic light scattering (herein after referred to as “DLS”) is used when the coacervates are less than a few microns and the solution conditions are dilute. In laser diffraction, the light scattered by the coacervates are measured by a series of detectors placed at different angles. The use of back scattering detectors and Mie theory enables detection of coacervate sizes less than 1 micron. The laser diffraction technique can be utilized to measure coacervates over a broader size range compared to DLS, and resolution of two populations of coacervates sizes (such as primary and colloidal particles) can be determined provided the difference in sizes is significant enough. In a focused beam reflectance measurement (FBRM), a chord length distribution, which is a “fingerprint” of the coacervate size distribution, is obtained. In FBRM, a focused laser beam scans across diluted composition in a circular path and the backscattered light is detected as pulses of light. The duration of the pulse is converted to a chord length, and by measuring thousands of chord lengths each second, the chord length distribution is generated. In FBRM, detection of two size populations can be obtained provided the differences in two size populations are great enough. FBRM is used when the coacervates are greater than approximately 1 micron and is particularly useful when the turbidity and/or coacervate concentration in solution is high. When measuring the coacervate particle size via scattering, the diluted sample is either placed in a cell for measurement in the instrument (DLS or laser diffraction) or the probe is placed directly into the vessel (FBRM).

Coacervate Rheology Method: The rheological properties of coacervates of the present personal care composition are measured by obtaining a coacervate and measuring its properties on a stress controlled rheometer using 8 millimeter flat plate geometry. Coacervate is obtained from the coacervate isolation method previously described, for each of the dilutions indicated herein when a coacervate forms at that dilution. When coacervate is present, sufficient coacervate should be obtained to measure its properties using an 8 mm flat plate geometry with 1,000 micron gap (i.e., at least about 100 mg is generally sufficient). The coacervate obtained is transferred onto the rheometer base plate, ensuring no supernatant is present, which may require wicking supernatant from the coacervate surface using a lint free wipe prior to adding to the baseplate. Excess coacervate is trimmed when the gap is 1500 microns, prior to obtaining the gap setting for the measurement, to avoid loading stress into the coacervate by the trimming process after the gap is obtained. The gap is obtained and the sample allowed to relax for 1 minute. A stress sweep is run logarithmically between 0.1-1000 Pa at an angular frequency of 100 radians/second, obtaining sufficient data points to obtain a reliable average in the linear viscoelastic region for G′ and G″. The linear viscoelastic region is defined as the stress range over which G′ is constant, i.e., independent of stress. G′ and G″, measured in units of Pa, are averaged over the linear viscoelastic region to obtain a result.

Color Measurement Method: Initial and final color measurements are made of porcine or in-vivo human skin using a HUNTERLAB™ spectra colorimeter in reflectance mode, using a 0° light source and 45° detector geometry. The colorimeter is calibrated with the appropriate black and white standards. Measurements are made before and after wash treatment. Three measurements are made each time and averaged to obtain a result. Values of L, a*, and b*, are obtained. L measures units of “Lightness”, a* measures values from red to green and b* measures values from yellow to blue.

In-vivo deposition evaluation method: One method of evaluating deposition from personal care composition prototypes is an in-vivo deposition evaluation method which comprises an in-vivo forearm wash protocol on human test subjects (hereinafter referred to as “panelists”) followed by measurement of the skin of panelists by a spectrophotometer. The spectrophotometer used in the In-vivo deposition evaluation method, is a colorimeter. A suitable colorimeter for this purpose is a colorimeter equipped for reflectance measurements were the specular component can be excluded, such as the Coloreye 7000A available from Gretag Macbeth. A computer is used in the in-vivo deposition evaluation method to control the colorimeter and collect data from the panelists and the colorimeter. The computer is outfitted with a Optiview Propalette 5.1 software package that has a macro for measuring X, Y, and Z values as defined for the CIE standard observer and converting them to values that describe the color dimensions black to white, red to green, and blue to yellow, respectively. The in-vivo deposition evaluation method requires a source of running water having a controlled temperature in the range of 35-38° C. The method requires 1 ml syringes to hold the compositions of the control sample and the test sample. The in-vivo deposition evaluation method requires wash puffs (hereinafter referred to as “puffs”) for applying the control sample and test sample onto the skin of the panelist. Two puffs are used per panelist, one for use with the control sample and another for use with the test sample.

The colorimeter calibration is verified with a background scan including both a positive white control and a negative black control. The panelist is instructed to conduct four colorimeter scans on both the left and right inner forearms prior to performing the wash protocol, each of the four scans being in a marked area to be treated with no rash or skin discolorations present. The panelist is instructed to complete the in-vivo wash protocol for the left forearm. The in-vivo wash protocol is summarized below. 15 minutes after the panelist completes the in-vivo wash protocol for the left forearm, the panelist is instructed to place the washed portion of the left forearm on colorimeter. Four scans are preformed on the left forearm using the colorimeter. Next, the panelist is instructed to complete the in-vivo wash protocol for the right forearm. 15 minutes after the panelist completes the in-vivo wash protocol for the right forearm, the panelist is instructed to place the washed portion of the right forearm on the colorimeter. Four scans are performed on the right forearm using the colorimeter.

The scans performed by the colorimeter determine the visible spectrum of the light reflected from the surface of the skin. The L value generated by the colorimeter measures the deposition of the benefit agents. The individual L values of control arm and test arm prior to and after the wash protocol has been performed are subtracted from one another to calculate the change in L value (ΔL) for the treated arm and the control arm. The mean and standard deviation of those values are then calculated and reported. A t-test is performed to determine statistical significance.

In-vivo wash protocol: The panelist obtains a sample comprising a syringe containing 1 ml of personal care composition (hereinafter referred to as “sample”) and a puff, the puff comprising a gathering of mesh nylon commonly used for washing, for the arm they are washing.

A measurement area of the forearm is marked with indelible ink, ensuring that washing encompasses the marked area. The panelist is instructed to saturate the puff with running water for 5 seconds. The panelist holds the puff in the hand of the forearm they are currently washing while wetting the forearm under running water for 5 seconds, letting water flow from the elbow to the wrist. After the forearm is wetted, the panelist dispenses the sample syringe into the wet hand opposite the forearm being washed. The panelist rubs the sample onto the forearm from the elbow to the wrist in continuous, circular strokes for 5 seconds. The panelist transfers any excess sample sticking to the hand by rubbing the palm of the hand along the edge of the arm. Without re-wetting the puff, the panelist lightly washes the inner forearm from the wrist to the elbow with the puff for 10 seconds in continuous, circular strokes. The sample should lather on the forearm and not appear streaky—if the forearm appears streaky the test is repeated with less pressure and faster rubbing. The panelist leaves the applied sample on the forearm for 15 seconds and then rinses the forearm with gentle, warm (105 F) water from inner elbow to wrist for 15 seconds. The panelist pats the forearm dry with a single paper towel, using no rubbing motion, and air-dries for the forearm for 30 seconds, then waits 15 minutes before evaluation using the colorimeter. The panelist washes both forearms by the wash procedure.

Evaluation of Reflectance Modification: If evaluating the change in reflectance/radiance, initial measurements of the skin surface is made with a gloss meter which measures units of gloss. The gloss meter is first set with both detector and light source at 85° from normal. The gloss meter is calibrated with appropriate reflection standard. Measurements of gloss are taken before and after the in-vivo wash protocol and Δ gloss is expressed as a percentage change.

Evaluation by Modification in Appearance by Human Perception: 15 minutes after performing the in-vivo wash protocol, 20 untrained panelists examine the skin surface topically treated with the personal care composition and the skin surface topically treated with only water. The panelists are asked to individually identify the skin surface comprising the desired skin surface modification or designate no perceived difference between the skin surfaces. A 60% positive identification by the panelists is regarded as the minimum criteria for successful deposition of the cosmetic active.

In-vitro Deposition Evaluation Method: The In-vitro Deposition Evaluation Method measures the deposition of benefit agents on a skin mimic. The method compares spectral data of the skin mimic surface before and after cleansing in an automated cleansing unit, such as the automated cleansing unit described in co-pending and co-assigned Multiphase Personal Care Composition With Enhanced Deposition, U.S. application Ser. No. 12/510,880 (filed Jul. 28, 2009) and In-Vitro Deposition Evaluation Method for Identifying Personal Care Compositions Which Provide Improved Deposition of Benefit Agents, U.S. application Ser. No. 12/511,034 (filed Jul. 28, 2009).

The In-vitro Deposition Evaluation Method uses two 96-well microplates (hereinafter referred to as “microplates”). Suitable 96-well microplates are commercially available from PerkinElmer and from VWR.com. For example, the SpectraPlate 96-MG from PerkinElmer has 8 rows and 12 columns with a well volume of 400 μl. The SpectraPlate 96-MG comprises the approximate dimensions of 14.6 mm in height, 127.8 mm in length and 85.5 mm in width. The SpectraPlate 96-MG has a well diameter of 7.15 mm, a well depth of 10.8 and a well to well spacing of 9.0 mm. A 96-well microplate is provided for containing the samples comprising the personal care composition in the Examples below

The In-vitro Deposition Evaluation Method uses approximately 1536 bodies for two microplates. Eight bodies carefully loaded into each of the 96 wells of the two microplates to ensure the same number is loaded into each well. Each body is a spherical stainless steel bearing that is approximately 2 mm in circumference. Each body comprises ferrometallic material. Suitable bodies are those available from WLB Antriebeselemente Gmbh, Scarrastrasse 12, D-68307 Mannheim, Germany.

The personal care compositions are prepared according to the description in the Example Section below. After the examples of the personal care compositions are prepared, control and test samples are prepared by (1) combining a personal care composition and distilled water and pre-diluting or (2) determining the dilution ratio and dispensing both the personal care composition and distilled water into the wells of the microplate and allow the samples to mix while being exposed to the automated washing process. For pre-dilution (1), the following steps are taken: For each sample, 90±0.09 grams of distilled water is dispensed into a mixing vessel. The mixing vessel is secured to the base of a mixer, such as a table top mixer from IKA, the mixer blades are adjusted into the distilled water within the mixing vessel about halfway from the top surface of the water so that 500 rpm stir speed creates a vortex that does not reach the blades. A syringe is then zeroed on a balance and then is filled with the designated personal care composition to slightly greater than 10 grams added composition. The mixer is turned on and a speed of 500 rpm is obtained, and 10 grams of the personal care composition is dispensed into the water within the mixing vessel. The distilled water and the designated personal care composition are mixed for 2 minutes at 500 rpm forming the sample. The sample is withdrawn by syringe from the mixing vessel while the mixer is on at a speed of 300 rpm. The mixing and dispensing procedures are followed for mixing and dispensing for the control sample and the test samples 1-5. After the samples are prepared, the control samples and test samples are dispensed in the specified wells of the microplate, all within a 20 minute time frame.

The skin mimic used in the In-vitro Deposition Evaluation Method is comprised of a molded bicomponent polyurethane substrate. The skin mimic is textured on one side with a pattern that resembles the texture of human skin The textured side of the skin mimic is coated with 1,1,1-trimethyl-1-pentene that is plasma deposited. The skin mimic surface has a total surface energy of 32±1.0 (mJ/m²) and a contact angle in water of 100°±2.0. Suitable skin mimic surface materials are described in co-pending and co-assigned Coated Substrate with Properties of Keratinous Tissue, U.S Patent Pub. No. 20070128255A1 (filed Aug. 11, 2006) (published Jun. 7, 2007) and Methods of Use of Substrate Having Properties of Keratinous Tissue, U.S Patent Pub. No. 20070288186A1 (filed Feb. 5, 2007) (published Dec. 13, 2007).

After all of the wells of the microplate are filled with the samples and the pieces of skin are made and coated, the skin mimic is prepared for the In-vitro Deposition Evaluation Method. Two pieces of skin mimic are prepared by cutting the skin mimic to fit on top of all 96 openings of the wells of the microplate while wearing gloves. The two pieces of skin mimic pieces are numbered “1” and “2”.

A base line spectral data is obtained by the spectrophotometer for both pieces of skin mimic. An Eye-one® IO Spectrophotometer from GretagMacbeth with Measure Tool Software (collectively hereinafter referred to as “spectrophotometer”) and a computer associated with the spectrophotometer (hereinafter referred to as “computer”) is utilized. The reading surface of the spectrophotometer is cleaned prior to each reading. The reading surface of the spectrophotometer is black in order to provide adequate sensitivity. The first piece of skin mimic is placed on the reading surface with the textured, treated region of the skin mimic facing the spectrophotometer. Next, a piece of plastic having a plurality of holes which correspond in size to the openings of the microplate is placed over the textured and treated region of the skin mimic A scan is then performed using the robot arm of the spectrophotometer. The baseline spectral data for the first piece of skin mimic is saved on a computer as the first baseline. The reading surface of the spectrophotometer is cleaned and the spectral data for the second piece of skin mimic surface is obtained, as described for the first piece of skin mimic. The baseline spectral data for the second piece of skin mimic is saved on the computer as the second baseline.

Next, the pieces of skin mimics are arranged over the openings of the wells of the microplates. The pieces of skin mimic surface material are transferred to cover the openings of the wells of the each of the microplates to ensure that the textured and treated region of the skin mimic is facing the openings of the wells of the microplate. A lid is placed over each piece of the skin mimic and the associated microplate to form a lidded microplate.

The lidded microplates are placed into microplate holders of an automated cleansing unit, or, a device used in the in-vitro Deposition Evaluation Method of the present invention. The automated cleansing unit comprises a horizontal base comprising four microplate holders. The horizontal base is made of rectangle of aluminum comprising the following approximate dimensions of ⅜ inch in height, fourteen inches in width and twenty seven inches in length. The automated cleansing unit further comprises two vertical supports comprised of aluminum with the approximate dimensions of one inch by two inches by ten and ¾ of an inch in height. The vertical supports are attached to a horizontal support comprising a rodless air slide. The horizontal support comprising a rodless air slide comprises the approximately dimension of a ½ inch by two inches by twenty six and ½ inches in height. Suitable rodless air slides comprise a one inch bore and eleven inch stroke and have associated end lugs and mount brackets, which are commercially available from McMaster-Carr. The rodless air slide can be double acting and comprises a carriage that is connected to an internal piston and two compressed air ports.

The automated cleansing unit comprises two magnetic arms. The horizontal support comprising a rodless air slide is the structure upon which the two magnetic arms are mounted. The magnetic arms are mounted to the rodless air slide such that the magnetic arms move back and forth along the length of the double acting rodless air slide by the force of compressed air. Each of the magnetic arms are comprised of aluminum and have the approximate dimensions of one inch by two inches by fourteen inches in length and have a “T” shape channel that houses seven neodymium iron boron magnets (not shown). Each of the neodymium iron boron magnets has the approximate dimensions of two inches in length, one inch in width and half or an inch in height. Each of the neodymium iron boron magnets comprises a magnetic strength of 12200 Gauss, available from Edmund Scientifics. The magnetic arms are configured at a height of about 2.75 cm above the microplate holder with the caveat that the magnets maintain their function to attract and move the bodies comprised within the wells of the microplate. The magnetic arms move back and forth along the length of the rodless air slide by the force of compressed air at a speed of approximately 6 back and forth sweeps over the length of the rodless air slide over a 10 second time period.

The magnetic arms can be configured with four microplate holders. Each of the microplate holders comprise a clamping plate and four pistons attached to a pneumatic control unit. When actuated, the pistons for the pneumatic control unit hold the microplates in the four microplate holders at a pressure of about 90 psi. Prior to placing the lidded microplates into the microplate holders of automated cleansing unit, the pneumatic control unit is turned on.

The automated cleansing unit can comprise a pneumatic control unit according to one embodiment. The top view shows components of the pneumatic control unit which can be connected to the rodless air slide, the piston and clamping plates. The pneumatic control unit can be used to apply compressed air to the automated cleansing unit, which imparts a force by converting the potential energy of compressed air into kinetic energy. The pneumatic control unit comprises a solenoid air control valve, a distribution manifold outlet, a compressed air control valve, a compressed air flow regulator, an alternating output binary valve, a two-hand safety pneumatic control valve, a compressed air control valve and various connectors that provide pressurized air to the automated cleansing unit from an external air source. The air control valve, air flow regulators, alternating a binary valves, a two-hand safety pneumatic control valve are positioned upstream of a solenoid air control valve. A suitable solenoid air control valve, in one embodiment, is described as a double air style valve with a 10 psi to 120 operating pressure. Suitable compressed air flow regulators, in some embodiments, operate in the pressure range of 14 psi to 116 psi. Suitable air control valve alternating output binary valves 40, in some embodiments, operate in a 35 psi to 100 psi range. All of the components of the pneumatic control unit are available from McMaster-Carr®.

The lidded microplates are placed into the microplate holders and pneumatic control unit is actuated such that the lidded microplates are held under 90 psi of pressure. The magnetic arms are actuated on and arms moves over the lidded microplates at a height of 2.65 cm above the microplate holders. The magnetic arms of the automated cleansing unit, sweep back and forth over the microplate holders for 5 minutes, at a speed of 6 sweeps per every 10 seconds. After 5 minutes of the automated cleansing process, the lidded microplates are removed from the microplate holders and are disassembled so that spectral data is gathered by a spectrophotometer for both pieces of skin mimic surface material.

After the automated washing process but prior to the final spectral readings, two large 4000 ml beakers of 20° C. to 25° C. water are filled. The first piece of skin mimic is removed from the first microplate and submerged in the tap water within the first beaker five times. The second piece of skin mimic is removed from the second microplate and submerged within the second beaker five times. The completeness of rinsing step is judged visually by the lack of foam on the skin mimic and presence of defined circles of deposited material on the skin mimic Both piece of skin mimic are blotted gently with paper towels and fumed in a drying hood for five minutes each. The reading surface of the spectrophotometer is cleaned. The first piece of skin mimic is placed on the reading surface with the textured and treated region of the first skin mimic facing the spectrophotometer. Next, a piece of plastic having a plurality of holes which correspond in size to the openings of the microplate is placed over the textured and treated region of the first skin mimic. The scan is then performed using the robot arm of the spectrophotometer. The spectral data for the first piece of skin mimic material is saved for comparison with the first baseline. The reading surface of the spectrophotometer is cleaned and the spectral data for the second piece of skin mimic surface material is obtained by the aforesaid method. The baseline spectral data for the second skin mimic surface material is saved on a computer for comparison with the second set of spectral data.

The spectrophotometer measures the L-a-b values for the skin mimic surface material before cleansing and after washing. The deposition values of the In-vitro Deposition Evaluation Method are reported as a Delta L value and are indicative of the deposition profile of each sample. The difference of the light intensity L or “Delta-L” is the L value after the cleansing−L value before cleansing (the baseline spectral data). The percent difference in Delta L is calculated and can be indexed relative to Delta L obtained for a control.

In-vitro deposition evaluation method for interference pigment: The skin mimic substrate used is that disclosed in commonly owned and assigned U.S. Publication No. 2007/012855 entitled “Coated Substrate with Properties of Keratinous Tissues.” The skin mimic was prepared for the experimentation by the steps disclosed. A cleansing puff is saturated with tap water having a temperature of 90°-95° and set aside. The a piece of the skin mimic substrate approximately 18 cm×7 cm is wet under tap water for 5 seconds and set aside. 1 cc of a personal care composition comprising an interference pigment is loaded into a small syringe. The skin mimic substrate is randomly dotted with the entire contents of the syringe of the personal care composition. The personal care composition is evenly distributed over the skin mimic substrate for 5 seconds by spreading with a gloved hand. Without re-wetting the puff, the skin mimic substrate is washed or 10 seconds in long, continuous, circular strokes, washing through the personal care composition with each stroke. The personal care composition should appear foamy on the skin mimic: if it is streaky, too much pressure is being applied. The personal care composition is allowed to remain on the skin mimic for 15 seconds after the washing step, and then rinsed for 15 seconds with tap water at ca. 1.5 gallons/min flow rate with flow directed indirectly to the treated surface. The water temperature is 95 F, +/−5 F. The skin mimic substrate is gently patted dry with a clean paper towel. The skin mimic is allowed to dry for 1 hour at ambient conditions drying before taking any particle count readings.

After the skin mimic substrate is treated and dried a particle count is performed to determine the amount of deposition of the interference pigments from the personal care composition onto the skin mimic. An optical microscope model Hi-Scope (HIROX Co. Ltd., Japan) or comparable is used at ˜×200 magnification. The skin mimic is equally divided into 12 sessions and the pigments in the center of each session are counted. The total number of the interference pigment particles on the twelve sites is used as the particle count for each skin mimic. Three replicate skin mimics are conducted for each composition and the average of the three swatches is used as the average count.

Lather Volume Test: Lather volume is measured using a graduated cylinder and a rotating mechanical apparatus. A 1,000 ml graduated cylinder is used which is marked in 10 ml increments, has a height of 14.5 inches at the 1,000 ml mark from the inside of its base, and has a neck at its top fitted for a plastic insert cap (for example, Pyrex No. 2982). Moderately hard water is prepared with 1.5:1 ion ratio Ca/Mg by dissolving 1.14 grams calcium chloride dihydrate and 1.73 grams magnesium chloride hexahydrate into one U.S. gallon distilled water. The water is maintained at between 105-110° F. The graduated cylinder is heated to about the same temperature by flushing with excess tap water at the same temperature for about 15 seconds, then drying it inside and out. 100.0 grams of the moderately hard water at the indicated temperature is weighed directly into the graduated cylinder. The cylinder is clamped in a mechanical rotating device, which clamps the cylinder vertically with an axis of rotation that transects the center of the graduated cylinder. Using a 3- or 4-place metric balance, invert the plastic cap for the graduated cylinder onto the balance pan and weigh 0.500 grams of composition to within 4 milligrams accuracy, using a holder to keep the cap level. Insert the cap into the graduated cylinder neck while being careful that all composition is now in the space in the cylinder interior. For compositions with very low viscosity which will not remain on the cap surface, 500 mg composition can be added directly to the graduated cylinder. Rotate the cylinder for 25 complete revolutions at a rate of about 10 revolutions per 18 seconds to create a lather and stop in a level, vertical position. A timer is set to allow 15 seconds for drainage. After 15 seconds, the lather volume is measured by recording the lather height to the nearest 10 ml mark (including any water that has drained to the bottom on top of which the lather is floating). The entire process should take less than 3 minutes in order to maintain desired temperature.

If the top surface of the lather is uneven, the lowest height at which it is possible to see halfway across the graduated cylinder is the lather volume (ml). If the lather is so coarse that a single or only a few foam cells (“bubbles”) reach across the entire cylinder, the height at which at least about 10 foam cells are required to fill the space is the lather volume, also in ml up from the base. In one embodiment, the maximum foam height is about 1,000 ml (even if the total foam height exceeds the 1,000 ml mark on the graduated cylinder). In an alternate embodiment, the maximum foam height is from about 200 ml to about 800 ml, alternatively from about 250 ml to about 700 ml, alternatively from about 300 ml to about 650 ml. The measurement is repeated and at least three results averaged to obtain the lather volume.

Gas chromatography-mass spectrometry (GC-MS) evaluation method for an enrichment ratio associated with perfumes: A ratio of a concentration of a perfume or a perfume raw material in a coacervate to that in a supernatant is measured using Liquid injection Gas Chromatography/Mass Spectrometry. For example, hexane extract solutions of perfumes or perfume raw materials are analyzed using a system such as a MPS2-Liquid-GC-MS analysis system (GC-02001-0153, MSD-02001-0154, MPS2-02001-0155). The system may include a Gas Chromatograph (GC): Agilent model 6890 with a CIS-4 injector (Gerstel, Mulheim, Germany), an MPS-2 Autosampler, and TDU (for liquid injection analysisa split/splitless injector can be used). The system may further include a GC column: J&W DB-5 MS, 30 M×0.25 mm ID, 1.0 μm film thickness obtained from J&W Scientific of Folsom, Calif., USA, a carrier gas such as helium at a 1.5 ml/min flow rate, an injector liner such as a liner with glass wool from Supelco, and a detector such as a model 5973 Mass Selective Detector obtained from Agilent Technologies, Inc., Wilmington, Del., USA having a source temperature of about 230° C., and a MS Quad temperature of about 150° C.

To measure the hexane extract solutions of a perfume or a perfume raw material, a sample of a hexane extract solution is transferred to a proper sample tray and the sample is analyzed using Liquid injection Gas Chromatography/Mass Spectrometry techniques. A sequence of sample loading and analysis is started with the injector temperature being set to 260° C. Then, GC-MS analysis run is started with the injection volume being 1 μl and an appropriate split ratio of, e.g., 0-25 being used based on the concentration of the perfume raw material in the extract. The following temperature program is used: an initial temperature of about 50° C. which is held for 3 minutes, increase the initial temperature at a rate of about 6° C./min until a temperature of about 250° C. is reached, then 25° C./min to 275° C., hold at about 275° C. for 4.67 minute.

The perfume or perfume raw material is subsequently identified using the MS spectral libraries of John Wiley & Sons and the National Institute of Standards and Technology (NIST), purchased and licensed through Hewlett Packard. Chromatographic peaks for specific ions are integrated using Chemstation software obtained from Agilent Technologies, Inc., Wilmington, Del., USA. The ratio for each perfume or perfume raw material is then measured as a ratio of peak area for the perfume or perfume raw material in coacervate vs. peak area for the perfume or perfume raw material in the supernatant, taking into account of the split ratios used in the injection. The average ratio or perfume enrichment ratio can then be calculated as the average of the ratios for all perfumes or perfume raw materials being measured.

### Examples

The following examples further describe and demonstrate embodiments within the scope of the invention. The examples are given solely for the purpose of illustration and are not to be construed as limitations of the present invention, as many variations thereof are possible without departing from the spirit and scope of the invention. All compositions shown are on an active material basis even if the ingredient is a dilution.

The examples are prepared by the following method. In a mixing vessel, add surfactants, disodium EDTA and sodium benzoate to water with agitation until sodium benzoate and EDTA are dissolved. The pH of the surfactants, disodium EDTA, sodium benzoate and water mixture is adjusted to a pH of 5.5 to 6.5 with citric acid or sodium citrate. Sodium chloride and remaining water are added to the mixing vessel and the mixture is mixed until thickened. The hydrophobic component, optical modifier and cationic acrylamide polymer are added to the mixing vessel with gentle agitation. If the cationic polymer is in solid form, it is pre-dissolved in water before addition. The resultant personal care composition is mixed using a DAC-150 SpeedMixer™ (from FlackTek Inc.) at 2000 rpm for 1 min.

TABLE 1
Personal care compositions comprising polymers with triquat and diquat monomers

| | Comparative Examples | | | Inventive Examples | | |
|---|---|---|---|---|---|---|
| Ingredients: | A | B | C | D | E | F |
| sodium laureth sulfate 3 mol ethoxylated (29%, | 6.8 | 6.8 | 6.8 | 6.8 | 6.8 | 6.8 |
| P & G Chemicals, Cincinnati, OH) | | | | | | |
| sodium lauryl sulfate (28%, P & G) | 2.6 | 2.6 | 2.6 | 2.6 | 2.6 | 2.6 |
| cocamidopropyl betaine (MIRATAINE ® | 1.0 | 1.0 | 1.0 | 1.0 | 1.0 | 1.0 |
| CAB/AS, Rhodia Inc.) | | | | | | |
| citric acid anhydrous | 0.16 | 0.16 | 0.16 | 0.16 | 0.16 | 0.16 |
| disodium EDTA (DISSOLVINE NA 2x from | 0.1 | 0.1 | 0.1 | 0.1 | 0.1 | 0.1 |
| Akzo Nobel) | | | | | | |
| sodium benzoate (PUROX ™ S Grains from | 0.26 | 0.26 | 0.26 | 0.26 | 0.26 | 0.26 |
| DSM N.V. Corp.) | | | | | | |
| methylchloroisothiazolinone and | 0.0005 | 0.0005 | 0.0005 | 0.0005 | 0.0005 | 0.0005 |
| methylisothiazolinone (KATHON ™ CG from | | | | | | |
| Rohm & Haas) | | | | | | |
| sodium chloride | 3.4 | 3.4 | 3.4 | 3.4 | 3.4 | 3.4 |
| titanium dioxide (RBTD-11S2 from KOBO | 2.0 | 2.0 | 2.0 | 2.0 | 2.0 | 2.0 |
| Products) | | | | | | |
| polyquaternium 76, COUG 5 | — | — | — | — | — | 0.3 |
| AM:TRIQUAT(95:5) (10% aq., Rhodia Inc., | | | | | | |
| Hillsborough, NJ, USA) | | | | | | |
| AM:TRIQUAT (80:20) | — | — | — | — | 0.3 | — |
| AM:TRIQUAT (97.5:2.5) | — | — | — | 0.3 | — | — |
| AM:DIQUAT (70:30) | — | — | 0.3 | — | — | — |
| AM:DIQUAT (92.5:7.5) | — | 0.3 | — | — | — | — |
| water | Q.S. | Q.S. | Q.S. | Q.S. | Q.S. | Q.S. |
| Dilution Ratio of personal care composition and | 1:9 | 1:9 | 1:9 | 1:9 | 1:9 | 1:9 |
| water for in-vitro deposition sample | | | | | | |
| Delta L index obtained from in-vitro deposition | 0.24 | 0.08 | 0.32 | 0.40 | 0.48 | 0.46 |
| evaluation method | | | | | | |

TABLE 2
Personal care compositions comprising random synthetic polymers
with various ratios of nonionic monomers to cationic monomers

| | Inventive | | Comparative | |
|---|---|---|---|---|
| | Examples | | Examples | |
| Ingredients: | F | F1 | F2 | F3 |
| sodium laureth sulfate 3 mol ethoxylated (P & G) | 6.8 | 6.8 | 6.8 | 6.8 |
| sodium lauryl sulfate (P & G) | 2.6 | 2.6 | 2.6 | 2.6 |
| cocamidopropyl betaine (MIRATAINE ® | 1.0 | 1.0 | 1.0 | 1.0 |
| CAB/AS) | | | | |
| citric acid anhydrous | 0.16 | 0.16 | 0.16 | 0.16 |
| disodium EDTA (DISSOLVINE NA 2x from | 0.1 | 0.1 | 0.1 | 0.1 |
| Akzo Nobel) | | | | |
| sodium benzoate (PUROX ™ S Grains from DSM | 0.26 | 0.26 | 0.26 | 0.26 |
| N.V. Corp.) | | | | |
| methylchloroisothiazolinone and | 0.0005 | 0.0005 | 0.0005 | 0.0005 |
| methyllisothiazolinone (KATHON ™ CG from | | | | |
| Rohm & Haas) | | | | |
| sodium chloride | 3.4 | 3.4 | 3.4 | 3.4 |
| titanium dioxide (RBTD-11S2 from KOBO | 2.0 | 2.0 | 2.0 | 2.0 |
| Products) | | | | |
| Polyquaternium 76, COUG 5 | 3.0 | | | |
| AM:TRIQUAT(95:5) (10%) | | | | |
| AM:TRIQUAT (70:30) | | 3.0 | | |
| AM:TRIQUAT (50:50) | | | 3.0 | |
| AM:TRIQUAT (10:90) | | | | 30 |
| Water | QS | QS | QS | QS |
| Dilution Ratio of personal care composition and | 1:9 | 1:9 | 1:9 | 1:9 |
| water for in-vitro deposition sample | | | | |
| Delta L index obtained from in-vitro deposition | 0.56 | 0.40 | 0.31 | 0.29 |
| evaluation method | | | | |

The Examples in Table 1 and Table 2 demonstrate the effect that the monomer ratio of the random synthetic polymer has on deposition of cosmetic actives. As shown in Table 1 and Table 1 the embodiments of the present invention have greater deposition of cosmetic actives versus control and the comparative examples. The data from Table 1 and Table 2 is also shown in FIG. 1 in the form of a bar chart. Table 1 illustrates enhanced deposition of cosmetic actives from a personal care composition comprising a random synthetic polymer comprising a triquat monomer versus the deposition from a personal care composition comprising a random synthetic polymer comprising a diquat monomer. Table 2 demonstrates the importance of the molar ratio of the acrylate monomer to cationic monomer for the random synthetic polymers on the deposition of cosmetic actives from a personal care composition.

TABLE 3
Personal care compositions comprising random synthetic polymers with
various ratios of nonionic monomers to cationic monomers and a
hydrophobic component

| | Comparative Examples | | | |
|---|---|---|---|---|
| Ingredients: | G | H | I | J |
| sodium laureth sulfate 3 mol ethoxylated (P & G) | 6.8 | 6.8 | 6.8 | 6.8 |
| sodium laureth sulfate 1 mol ethoxylated (P & G) | | | | |
| sodium lauryl sulfate (Procter & Gamble) | 2.6 | 2.6 | 2.6 | 2.6 |
| cocamidopropyl betaine (MIRATAINE ® CAB/AS) | 1.0 | 1.0 | 1.0 | 1.0 |
| citric acid anhydrous | 0.16 | 0.16 | 0.16 | 0.16 |
| disodium EDTA (DISSOLVINE NA 2x from Akzo | 0.1 | 0.1 | 0.1 | 0.1 |
| Nobel) | | | | |
| sodium benzoate (PUROX ™ S Grains from DSM | 0.26 | 0.26 | 0.26 | 0.26 |
| N.V. Corp.) | | | | |
| methylchloroisothiazolinone and | 0.0005 | 0.0005 | 0.0005 | 0.0005 |
| methylisothiazolinone (KATHON ™ CG) | | | | |
| sodium chloride | 3.4 | 3.4 | 3.4 | 3.4 |
| titanium dioxide (RBTD-11S2 from Kobo Products) | 2.0 | 2.0 | 2.0 | 2.0 |
| castor oil (Crystal LC USP, G.R. OShea Co., Itasca, | 1.0 | 1.0 | 1.0 | 1.0 |
| IL, USA) | | | | |
| AM:TRIQUAT (97.5:2.5) | — | — | — | 0.3 |
| AM:DIQUAT (70:30) | — | — | 0.3 | — |
| AM:DIQUAT (92.5:7.5) | — | 0.3 | — | — |
| water | Q.S. | Q.S. | Q.S. | Q.S. |
| Dilution Ratio of personal care composition and | 1:9 | 1:9 | 1:9 | 1:9 |
| water for in-vitro deposition sample | | | | |
| Delta L index obtained from in-vitro deposition | 0.34 | 0.12 | 0.29 | 0.26 |
| evaluation method | | | | |

TABLE 4
Personal care compositions comprising random synthetic polymers with various ratios of
cosmetic active, the cationic polymer and a hydrophobic component.

| | Inventive Examples | | | | |
|---|---|---|---|---|---|
| Ingredients: | K | L | M | N | O* |
| sodium laureth sulfate 3 mol ethoxylated (P & G) | 6.8 | 6.8 | 6.8 | 6.8 | — |
| sodium laureth sulfate 1 mol ethoxylated (P & G) | | | | | 4.4 |
| sodium lauryl sulfate (Procter & Gamble) | 2.6 | 2.6 | 2.6 | 2.6 | 4.4 |
| cocamidopropyl betaine (MIRATAINE ® | 1.0 | 1.0 | 1.0 | 1.0 | — |
| CAB/AS) | | | | | |
| GENAGEN KB (Clariant Functional Chemicals, | — | — | — | — | 1.25 |
| Switzerland) | | | | | |
| citric acid anhydrous | 0.16 | 0.16 | 0.16 | 0.16 | 0.22 |
| disodium EDTA (DISSOLVINE NA 2x from | 0.1 | 0.1 | 0.1 | 0.1 | 0.1 |
| Akzo Nobel) | | | | | |
| sodium benzoate (PUROX ™ S Grains from DSM | 0.26 | 0.26 | 0.26 | 0.26 | 0.25 |
| N.V. Corp.) | | | | | |
| methylchloroisothiazolinone, | 0.0005 | 0.0005 | 0.0005 | 0.0005 | 0.0005 |
| methylisothiazolinone (KATHON ™ CG from | | | | | |
| Rohm & Haas) | | | | | |
| sodium chloride | 3.4 | 3.4 | 3.4 | 3.4 | 3.4 |
| titanium dioxide (RBTD-11S2) | 2.0 | 2.0 | 2.0 | 0.5 | 2.0 |
| castor oil (Crystal LC USP) | 1.0 | 1.0 | 1.0 | 0.25 | 1.0 |
| polyquaternium 76, COUG 5 (95:5) | 0.3 | 0.3 | 0.05 | 0.10 | 0.30 |
| AM:TRIQUAT (10% aq.) | | | | | |
| Water | Q.S. | Q.S. | Q.S. | Q.S. | Q.S. |
| Dilution Ratio of personal care composition and | 1:9 | 1:9 | 1:9 | 1:9 | 1:3 |
| water for in-vitro deposition sample | | | | | |
| Delta L index obtained from in-vitro deposition | 0.65 | 1.20 | 1.04 | 0.99 | 1.19 |
| evaluation method | | | | | |

TABLE 5
Personal care compositions comprising random synthetic polymers with
various ratios of nonionic monomers to cationic monomers and a
hydrophobic component.

| | Inventive Examples | | | |
|---|---|---|---|---|
| Ingredients: | K | L 1 | L2 | L3 |
| sodium laureth sulfate 3 mol ethoxylated (P & G) | 6.8 | 6.8 | 6.8 | 6.8 |
| 2.6sodium lauryl sulfate (Procter & Gamble) | 2.6 | 2.6 | 2.6 | 2.6 |
| cocamidopropyl betaine (MIRATAINE ® CAB/AS) | 1.0 | 1.0 | 1.0 | 1.0 |
| GENAGEN KB liquid | | | | |
| citric acid anhydrous | 0.16 | 0.16 | 0.16 | 0.16 |
| disodium EDTA (DISSOLVINE NA 2x from Akzo Nobel) | 0.1 | 0.1 | 0.1 | 0.1 |
| sodium benzoate (PUROX ™ S Grains from DSM N.V. Corp.) | 0.26 | 0.26 | 0.26 | 0.26 |
| methylchloroisothiazolinone, methyllisothiazolinone | 0.0005 | 0.0005 | 0.0005 | 0.0005 |
| (KATHON ™ CG from Rohm & Haas) | | | | |
| sodium chloride | 3.4 | 3.4 | 3.4 | 3.4 |
| titanium dioxide (RBTD-11S2 from Kobo Products) | 2.0 | 2.0 | 2.0 | 2.0 |
| castor oil (Crystal LC USP) | 1.0 | 1.0 | 1.0 | 1.0 |
| AM:TRIQUAT (80:20) | 0.3 | | | |
| AM:TRIQUAT (70:30) | | 0.3 | | |
| AM:TRIQUAT (50:50) | | | 0.3 | |
| AM:TRIQUAT (10:90) | | | | 0.3 |
| water | Q.S. | Q.S. | Q.S. | Q.S. |
| Dilution Ratio of personal care composition and water for in- | 1:9 | 1:9 | 1:9 | 1:9 |
| vitro deposition sample | | | | |
| Delta L index obtained from in-vitro deposition evaluation | 0.59 | 0.41 | 0.56 | 0.44 |
| method | | | | |

TABLE 6
Personal care compositions comprising random synthetic
polymers and a hydrophobic component and a perfume

| | Comparative | Inventive | |
|---|---|---|---|
| | Example | Example | |
| Ingredients: | MM | NN | OO |
| sodium laureth sulfate 3 mol | 6.8 | 6.8 | 6.8 |
| ethoxylated (P & G) | | | |
| sodium lauryl sulfate (Procter & | 2.6 | 2.6 | 2.6 |
| Gamble) | | | |
| cocamidopropyl betaine | 1.0 | 1.0 | 1.0 |
| (MIRATAINE ® CAB/AS) | | | |
| citric acid anhydrous | 0.16 | 0.16 | 0.16 |
| disodium EDTA (DISSOLVINE NA 2x | 0.1 | 0.1 | 0.1 |
| from Akzo Nobel) | | | |
| sodium benzoate (PUROX ™ S Grains | 0.26 | 0.26 | 0.26 |
| from DSM N.V. Corp.) | | | |
| methylchloroisothiazolinone and | 0.0005 | 0.0005 | 0.0005 |
| methylisothiazolinone | | | |
| (KATHON ™ CG) | | | |
| sodium chloride | 3.4 | 3.4 | 3.4 |
| castor oil (Crystal LC USP, G.R. OShea | — | — | 1.0 |
| Co., Itasca, IL, USA) | | | |
| polyquaternium 76, COUG 5 (95:5) | — | 0.3 | 0.3 |
| AM:TRIQUAT (10% aq.) | | | |
| Perfume | 1.0 | 1.0 | 1.0 |
| water | Q.S. | Q.S. | Q.S. |
| In-vitro deposition of Perfume | 1.00 | | 1.60 |
| Enrichment ratio | 0 | 7 | 26 |

The Examples in Table 3, Table 4 Table 5, and Table 6 demonstrate greater enhancement of deposition with the addition of a hydrophobic component in personal care compositions of the present invention. As shown in Table 3, Table 4 and Table 5, the embodiments of the present invention have greater deposition of cosmetic actives versus control and the comparative examples. The data from Table 3, Table 4 and Table 5, is also shown in FIG. 2 in the form of a bar chart. Additionally, as shown in Table 6, embodiments of the present invention have greater deposition and encapsulation of a cosmetic active that is a perfume versus control and the comparative examples.

TABLE 7
Personal care compositions comprising random synthetic polymers, a
hydrophobic component and both coated and uncoated optical modifiers

| | Inventive | Comparative | Inventive | |
|---|---|---|---|---|
| | Example | Examples | Examples | |
| Ingredients: | L | P | Q | R |
| sodium laureth sulfate 3 mol ethoxylated (Procter | 6.8 | 6.8 | 6.8 | 6.8 |
| & Gamble) | | | | |
| sodium lauryl sulfate (Procter & Gamble) | 2.6 | 2.6 | 2.6 | 2.6 |
| cocamidopropyl betaine (MIRATAINE ® | 1.0 | 1.0 | 1.0 | 1.0 |
| CAB/AS) | | | | |
| citric acid anhydrous | 0.16 | 0.16 | 0.16 | 0.16 |
| disodium EDTA (DISSOLVINE NA 2x from | 0.1 | 0.1 | 0.1 | 0.1 |
| Akzo Nobel) | | | | |
| sodium benzoate (PUROX ™ S Grains from DSM | 0.26 | 0.26 | 0.26 | 0.26 |
| N.V. Corp.) | | | | |
| methylchloroisothiazolinone, | 0.0005 | 0.0005 | 0.0005 | 0.0005 |
| methylisothiazolinone (KATHON ™ CG from | | | | |
| Rohm & Haas) | | | | |
| Sodium chloride | 3.4 | 3.4 | 3.4 | 3.4 |
| castor oil (Crystal LC USP) | 1.0 | — | — | 1.0 |
| titanium dioxide (KOBO RBTD, no coating) | — | 2.0 | 2.0 | 2.0 |
| titanium dioxide (RBTD-11S2 from KOBO | 2.0 | | | |
| Products) | | | | |
| polyquaternium 76, COUG 5 (95:5) | 0.3 | — | 0.3 | 0.3 |
| AM:TRIQUAT (10% aq.) | | | | |
| water | Q.S. | Q.S. | Q.S | Q.S. |
| Dilution Ratio of personal care composition and | 1:9 | 1:9 | 1:9 | 1:9 |
| water for in-vitro deposition sample | | | | |
| Delta L index obtained from in-vitro deposition | 1.20 | 0.19 | 0.86 | 1.08 |
| evaluation method | | | | |
| % human noticeability of skin whiteness | 100 | — | — | — |
| comparison vs. S | | | | |

In Table 7 above, Examples L, P, Q and R demonstrate that the deposition of cosmetic actives from the compositions of the present invention are not significantly effected by the coating of the optical modifier.

TABLE 8
Personal care compositions comprising random synthetic polymers,
a hydrophobic component and various levels of optical modifiers

| | Comparative | | | |
|---|---|---|---|---|
| | Examples | Inventive Examples | | |
| Ingredients: | S | T | U | V |
| sodium laureth sulfate 3 mol ethoxylated (P & G) | 6.8 | 6.8 | 6.8 | 6.8 |
| sodium lauryl sulfate (Procter & Gamble) | 2.6 | 2.6 | 2.6 | 2.6 |
| cocamidopropyl betaine (MIRATAINE ® | 1.0 | 1.0 | 1.0 | 1.0 |
| CAB/AS) | | | | |
| citric acid anhydrous | 0.16 | 0.16 | 0.16 | 0.16 |
| disodium EDTA (DISSOLVINE NA 2x) | 0.1 | 0.1 | 0.1 | 0.1 |
| sodium benzoate (PUROX ™ S Grains) | 0.26 | 0.26 | 0.26 | 0.26 |
| methylchloroisothiazolinone, | 0.0005 | 0.0005 | 0.0005 | 0.0005 |
| methylisothiazolinone (KATHON ™ CG from | | | | |
| Rohm & Haas) | | | | |
| sodium chloride | 3.4 | 3.4 | 3.4 | 3.4 |
| titanium dioxide (RBTD-11S2 from KOBO | | 0.25 | 0.50 | 1.00 |
| Products) | | | | |
| polyquaternium 76, COUG 5 (95:5) | | 0.3 | 0.3 | 0.3 |
| AM:TRIQUAT (10% aq.) | | | | |
| castor oil (Crystal LC USP) | | 0.13 | 0.26 | 0.50 |
| water | Q.S. | Q.S. | Q.S. | Q.S. |
| Dilution of personal care composition and water | 1:9 | 1.9 | 1:9 | 1:9 |
| for in-vitro deposition sample | | | | |
| Delta L index obtained from in-vitro deposition | 0 | 0.25 | 0.56 | 0.82 |
| evaluation method | | | | |
| % human noticeability of skin whiteness | 7.5 | — | — | 77.5 |
| comparison, V vs. S | | | | |

In Table 8, Examples T, U, V in Table demonstrate that the deposition of cosmetic actives from the compositions of the present invention correlates with an increase in concentration of optical modifier within the personal care composition.

TABLE 9
Personal care compositions comprising random synthetic polymers,
a hydrophobic component, and high surfactant component concentrations

| | Comparative | | Inventive |
|---|---|---|---|
| | Examples | | Examples |
| Ingredients: | W | X | Y |
| sodium lauryl sulfate (P&G) | 7.02 | 7.02 | 7.02 |
| sodium lauroamphoacetate (DEHYTON ® ML | 4.19 | 4.19 | 4.19 |
| from Cognis) | | | |
| sodium trideceth sulfate (P&G Chemicals | 7.02 | 7.02 | 7.02 |
| Kansas City ST3S) | | | |
| sodium chloride | 4.13 | 4.13 | 4.13 |
| fragrance | 1.54 | 1.54 | 1.54 |
| trideceth-3 (ICONOL ™ TDA-3I from BASF | 1.70 | 1.70 | 1.70 |
| Corp.) | | | |
| methylchloroisothiazolinone, | 0.0004 | 0.0004 | 0.0004 |
| methylisothiazolinone (KATHON ™ CG) | | | |
| citric acid anhydrous | 0.76 | 0.76 | 0.76 |
| guar hydroxypropyltrimonium chloride (N- | 0.51 | 0.51 | 0.51 |
| HANCE ® 3196 from Aqualon) | | | |
| xanthan gum (Keltrol 1000, Kelco, Inc, USA) | 0.19 | 0.19 | 0.19 |
| acrylonitrile/methacrylonitrile/methyl | 0.31 | 0.31 | 0.31 |
| methacrylate copolymer, isopentane (Wetted | | | |
| EXPANCEL ® 920 WE 40 d24) | | | |
| sodium benzoate (Purox Grains) | 0.17 | 0.17 | 0.17 |
| PEG-90M (POLYOX WSR 301) | 0.13 | 0.13 | 0.13 |
| disodium EDTA (DISSOLVINE NA 2x from | 0.13 | 0.13 | 0.13 |
| Akzo Nobel) | | | |
| Iso Jojoba Butter 35 (Simmondsia chinensis) | 0.33 | 0.33 | 0.33 |
| titanium dioxide (RBTD-11S2 from Kobo | 4.0 | 4.0 | 4.0 |
| Products) | | | |
| castor oil (Crystal LC USP) | | 2.0 | 2.0 |
| polyquaternium 76, COUG 5 (95:5) | — | — | 0.60 |
| AM:TRIQUAT (10% aq.) | | | |
| water | Q.S. | Q.S. | Q.S. |
| Dilution of personal care composition and | 1:19 | half dosage | concentrated |
| water for in-vitro deposition sample | dilution | | product form (2X) |
| | ratio | | |
| Delta L index obtained from in-vitro deposition | 0.30 | 0.09 | 1.44 |
| evaluation method | | | |

In Table 9, Examples W, X and Y above demonstrate that the deposition of cosmetic actives from the compositions of the present invention increase when the surfactant component is structured within the personal care composition.

TABLE 10
Personal care compositions of the present invention comprising interference pigments

| | | Inventive Examples | | | |
|---|---|---|---|---|---|
| Ingredients: | | AA | BB | CC | DD |
| sodium laureth sulfate 3 mol ethoxylated (P & G) | | 6.7 | 6.7 | 6.7 | 6.7 |
| sodium lauryl sulfate (Procter & Gamble) | | 2.6 | 2.6 | 2.6 | 2.6 |
| cocamidopropyl betaine (MIRATAINE ® | | 1.1 | 1.1 | 1.1 | 1.1 |
| CAB/AS) | | | | | |
| citric acid anhydrous | | 0.16 | 0.16 | 0.16 | 0.16 |
| disodium EDTA (DISSOLVINE NA 2x from | | 0.1 | 0.1 | 0.1 | 0.1 |
| Akzo Nobel) | | | | | |
| sodium benzoate (PUROX ™ S Grains from DSM | | 0.26 | 0.26 | 0.26 | 0.26 |
| N.V. Corp.) | | | | | |
| methylchloroisothiazolinone, | | 0.0004 | 0.0004 | 0.0004 | 0.0004 |
| methyllisothiazolinone (KATHON ™ CG) | | | | | |
| sodium chloride | | 3.3 | 3.3 | 3.3 | 3.3 |
| KTZ Vibrant Gold 11S10 (from KOBO) | | 0.6 | 0.6 | 0.2 | 0.4 |
| Triethoxycaprylylsilane | | | | | |
| Polyquaternium 76, COUG 5 (95:5) | | — | 0.3 | 0.1 | 0.2 |
| AM:TRIQUAT (10% aq.) | | | | | |
| water | | Q.S. | Q.S. | Q.S. | Q.S. |
| Particle count from in-vitro deposition evaluation | | 6 | 40 | 16 | 51 |
| method for interference pigment | | | | | |
| % human noticeability on a | AA vs. CC | 0 | — | 100 | — |
| product with more sparkle under | CC vs. DD | | | 0 | 100 |
| direct and indirect light. | | | | | |

In Table 10, Examples AA, BB, CC and DD demonstrate that the deposition of interference pigments as the cosmetic active within the composition. Examples AA, BB, CC and DD in Table 9 above also demonstrate that the deposition of cosmetic actives from the compositions of the present invention correlates with an increase in concentration of optical modifier within the personal care composition.

TABLE 11
Personal care compositions of the present invention
comprising various hydrophobic components

| | Inventive Examples | | | | |
|---|---|---|---|---|---|
| Ingredients | Z | Z1 | Z2 | Z3 | Z4 |
| Sodium Laureth Sulfate (SLE3S) | 6.82 | 6.82 | 6.82 | 6.82 | 6.82 |
| Sodium Lauryl Sulfate | 2.62 | 2.62 | 2.62 | 2.62 | 2.62 |
| Cocamidopropyl Betaine (Mirataine | 1.05 | 1.05 | 1.05 | 1.05 | 1.05 |
| CAB/AS) | | | | | |
| Citric Acid Anhydrous | 0.16 | 0.16 | 0.16 | 0.16 | 0.16 |
| Disodium EDTA (Dissolvine Na2-S) | 0.10 | 0.10 | 0.10 | 0.10 | 0.10 |
| Sodium Benzoate (Purox S Grains) | 0.26 | 0.26 | 0.26 | 0.26 | 0.26 |
| Methylchloroisothiazolinone (and) | 0.0005 | 0.0005 | 0.0005 | 0.0005 | 0.0005 |
| Methyllisothiazolinone (Kathon CG) | | | | | |
| Sodium chloride | 3.40 | 3.40 | 3.40 | 3.40 | 3.40 |
| Trihydroxystearin (Thixcin) | 0.30 | 0.30 | 0.30 | 0.30 | 0.30 |
| Titanium dioxide (KOBO RBTD-11S2) | 2.00 | 2.00 | 2.00 | 2.00 | 2.00 |
| Polyquaternium 76, COUG 5 (95:5) | 0.30 | 0.30 | 0.30 | 0.30 | 0.30 |
| AM:TRIQUAT (10%) | | | | | |
| Castor oil (Crystal LC USP) | | 1.00 | | | |
| Mineral oil (Hydrobright 1000) | | | 1.00 | | |
| Polybutylene | | | | 1.00 | |
| Butylene glycol | | | | | 1.0 |
| Water | Q.S. | Q.S. | Q.S. | Q.S. | Q.S. |
| TiO2 deposition, ΔL index | 1.53 | 1.59 | 1.49 | 1.73 | 1.65 |

TABLE 12
Personal care compositions of the present invention comprising a hydrophobic
component and colloidal suspending agents

| | Inventive Examples | | | | | |
|---|---|---|---|---|---|---|
| Ingredients: | EE | FF | GG | HH | II | JJ |
| sodium lauryl sulfate 3 mol ethoxylated | 7.80 | 7.80 | 7.80 | 7.80 | 7.80 | 7.80 |
| (P & G) | | | | | | |
| sodium lauryl sulfate (P & G) | 3.00 | 3.00 | 3.00 | 3.00 | 3.00 | 3.00 |
| cocamidopropyl betaine (MIRATAINE ® | 1.20 | 1.20 | 1.20 | 1.20 | 1.20 | 1.20 |
| CAB/AS) | | | | | | |
| citric acid anhydrous | 0.22 | 0.22 | 0.22 | 0.22 | 0.22 | 0.22 |
| trihydroxystearin (Thixcin ® from Rheox, | — | 0.30 | | 0.30 | 0.30 | 0.50 |
| Inc.) | | | | | | |
| disodium EDTA (DISSOLVINE NA 2x | 0.10 | 0.10 | 0.10 | 0.10 | 0.10 | 0.10 |
| from Akzo Nobel) | | | | | | |
| sodium benzoate (PUROX ™ S Grains | 0.25 | 0.25 | 0.25 | | | |
| from DSM N.V. Corp.) | | | | | | |
| methylchloroisothiazolinone, | 0.0005 | 0.0005 | 0.0005 | 0.0005 | 0.0005 | 0.0005 |
| methyllisothiazolinone (KATHON ™ CG | | | | | | |
| from Rohm & Haas) | | | | | | |
| sodium chloride | 2.50 | 2.50 | 2.50 | 2.50 | 2.50 | 2.50 |
| titanium dioxide (RBTD-11S2 from | 2.00 | 2.00 | 2.00 | 2.00 | 4.00 | 4.00 |
| KOBO Products) | | | | | | |
| mineral oil (HYBROBRITE ® 1000 from | — | — | 1.00 | 1.00 | 1.00 | 1.00 |
| Sonnerbonn) | | | | | | |
| Polyquaternium 76, COUG 5 | 0.30 | 0.30 | 0.30 | 0.30 | 0.30 | 0.30 |
| AM:TRIQUAT(95:5) (10%) | | | | | | |
| water | Q.S. | Q.S. | Q.S. | Q.S. | Q.S. | Q.S. |
| Dilution of personal care composition and | 1:9 | 1:9 | 1:9 | 1:9 | 1:9 | 1:9 |
| water for in-vitro deposition sample | | | | | | |
| Delta L index obtained from in-vitro | 0.56 | 1.26 | 0.44 | 0.67 | 1.04 | 1.48 |
| deposition evaluation method | | | | | | |
| Yield Stress | — | 0.96 | — | 0.93 | 1.2 | 1.3 |
| Lather Volume | 310 | 220 | 572 | 480 | 460 | 460 |
| Delta L obtained from in-vivo deposition | | 1.2 | | | 1.5 | |
| evaluation method and colorimeter | | | | | | |
| evaluation | | | | | | |
| % human noticeability of skin whiteness | | | | | 66% | 69% |
| comparison vs. S | | | | | | |

In Table 12, Examples EE, FF, GG, HH, II and JJ in Table 12 above demonstrate that the deposition of cosmetic actives is increased when a colloidal suspending agent is added to the personal cleansing composition. Examples EE, FF, GG, HH, II and JJ in Table 12 also demonstrate that the addition of the colloidal suspending agent does not adversely affect the yield stress or the lather volume of the composition.

TABLE 13
Personal care compositions comprising random synthetic
polymers with and without zinc pyrithione.

| | Comparative | Inventive |
|---|---|---|
| | Example | Example |
| Ingredients: | KK | LL |
| sodium laureth sulfate 1 mol ethoxylated | 6.1 | 6.1 |
| (P & G) | | |
| cocamidopropyl betaine | 2.4 | 2.4 |
| (MIRATAINE ® CAB/AS) | | |
| citric acid anhydrous | 0.25 | 0.25 |
| disodium EDTA (DISSOLVINE NA 2x from | 0.1 | 0.1 |
| Akzo Nobel) | | |
| sodium benzoate (PUROX ™ S Grains | 0.25 | 0.25 |
| from DSM N.V. Corp.) | | |
| methylchloroisothiazolinone, | 0.0003 | 0.0003 |
| methylisothiazolinone (KATHON ™ | | |
| CG from Rohm & Haas) | | |
| sodium chloride | 0.5 | 0.5 |
| Zinc pyrithione (Arch chemicals, Inc.) | — | 1.0 |
| polyquaternium 76, COUG 5 (95:5) | 0.30 | 0.30 |
| AM:TRIQUAT (10% aq.) | | |
| Water | Q.S. | Q.S. |
| Dilution Ratio of personal care composition | 1:3 | 1:3 |
| and water for in-vitro deposition sample | | |
| Delta L index obtained from in-vitro | 0 | 0.70 |
| deposition evaluation method | | |

In Table 13, Examples KK and LL demonstrate that a cosmetic active such antimicrobials including ZPT is deposited effectively from the personal care composition of the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a. a synthetic random polymer comprising a net positive charge; said synthetic random polymer comprising:
i. a acrylamide monomer unit of the following formula: 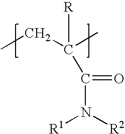
where R, R¹ and R² are a hydrogen; and
ii. a cationic monomer unit comprising 3 or more positive charges; said cationic monomer unit of the following formula: 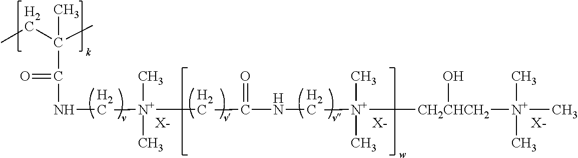
where k is 1, each of v, v′, and v″ independently comprise an integer from 1 to 4, w comprises an integer from 1 to 10, and X⁻ comprises a chloride anion;
wherein said synthetic random polymer comprises a ratio of said acrylamide monomer unit to said cationic monomer unit comprising from about 55:45 to about 97:3; and
b. an anionic surfactant component;
c. one or more water insoluble cosmetic actives; and
d. an aqueous carrier;
wherein upon dilution said personal care composition comprises one or more coacervates.

2. The personal care composition of claim 1, wherein said personal care composition comprises from about 0.05% to about 5.0%, by weight of said personal care composition, of said synthetic random polymer.

3. The personal care composition of claim 1, wherein said personal care composition comprises a lather volume from about 200 ml to about 750 ml.

4. The personal care composition of claim 1, wherein said one or more coacervates comprise a chord length of from about 30 μm to about 2 mm

5. The personal care composition of claim 1, wherein said one or more coacervates comprise an elastic modulus of about 100 Pa to 20,000 Pa.

6. The personal care composition of claim 1 further comprising from about 0.05% to about 10.0%, of said personal care composition selected from silicone oils, hydrocarbon oils, polyolefins, fatty esters and mixtures thereof.

7. A personal care composition comprising:
a. a synthetic random polymer comprising a net positive charge; said synthetic random polymer comprising:
i. a acrylamide monomer unit of the following formula: 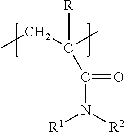
where R, R¹ and R² are a hydrogen; and
ii. a cationic monomer unit comprising 3 or more positive charges; said cationic monomer unit of the following formula: 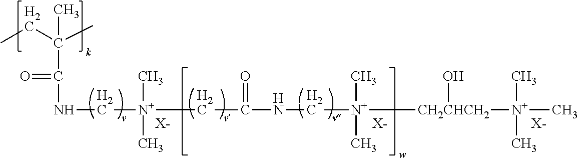
where k is 1, each of v, v′, and v″ independently comprise an integer from 1 to 4, w comprises an integer from 1 to 10, and X⁻ comprises a chloride anion;
wherein said synthetic random polymer comprises a molar ratio of said acrylamide monomer unit to said cationic monomer unit comprising from about 10:90 to about 97:3; and
b. a surfactant component;
c. a hydrophobic component:
d. one or more cosmetic actives; and
e. an aqueous carrier;
wherein upon dilution said personal care composition comprises one or more coacervates.

8. The personal care composition of claim 7, wherein said personal care composition comprises from about 0.20% to about 0.50%, by weight of said personal care composition, of said synthetic random polymer.

9. The personal care composition of claim 7, wherein said molar ratio of said acrylamide monomer unit to said cationic monomer unit comprises from about 80:20 to about 96.5:3.5.

10. The personal care composition of claim 7, wherein said molar ratio of said acrylamide monomer unit to said cationic monomer unit comprises 95:5.

11. The personal care composition of claim 7, wherein said personal care composition comprises from about 5% to about 22%, by weight of said personal care composition of said surfactant component selected from an anionic surfactant, an amphoteric surfactant, a zwitterionic surfactant, and mixtures thereof having an anionic charge.

12. The personal care composition of claim 7, wherein said personal care composition comprises from about 5% to about 15%, by weight of said personal care composition of said surfactant component selected from ethoxylated anionic comprising an ethoxylation level of from about 1 to about 3, a non-ethoxylated anionic surfactant, an amphoteric surfactant, a zwitterionic surfactant and mixtures thereof.

13. The personal care composition of claim 12, wherein said amphoteric or zwitterionic surfactant is selected from the group consisting of cocoamidopropyl betaine, coco betaine, cocoamphoacetate, cocoamphodiacetate, lauroamphoacetate, lauroamphodiacetate, lauramine oxide and mixtures thereof.

14. The personal care composition of claim 7, wherein said personal care composition comprises from about 0.1% to about 10% of said hydrophobic component selected from castor oil, mineral oil, polybutene, jojoba oil, silicone oils, petrolatum, triglyceride oils, gelled mineral oils, gelled vegetable oils, oils comprising crystalline structurants, oils comprising non-crystalline structurants and mixtures thereof.

15. The personal care composition of claim 7, wherein said personal care composition further comprises from about 0.01% to about 2.0%, by weight of the personal care composition, of a colloidal suspending agent selected from trihydroxystearin, ethylene glycol distearate, acyl derivatives, long chain amine oxides and mixtures thereof.

16. The personal care composition of claim 7, wherein said personal care composition further comprises from about 0.1% to about 10% of one or more cosmetic actives selected from optical modifiers; antimicrobials; perfumes; deodorant actives; vitamins; vitamin derivatives; sunscreens; desquamation actives; zinc carbonate; anti-wrinkle actives; anti-atrophy actives; anti-oxidants; skin soothing agents; skin healing agents; anti-acne medicaments; medicaments; essential oils; sensates; clays; and mixtures thereof.

17. The personal care composition of claim 7, wherein said dilution is based on a dilution ratio, and wherein the dilution ratio is between 1:0.1 and 1:50.

18. A method of treating a skin surface resulting in a modification in appearance of said skin surface; said method composition the steps of:
a. topically applying a personal care composition to said skin surface; said personal care composition comprising:
i. a synthetic random polymer comprising a net positive charge; said synthetic random polymer comprising:
1. a acrylamide monomer unit of the following formula: 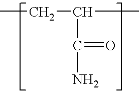
2. a cationic monomer unit comprising 3 or more positive charges;
said cationic monomer unit of the following formula: 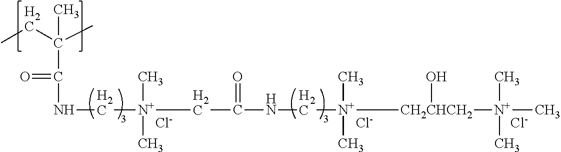
wherein said synthetic random polymer comprises a molar ratio of said acrylamide monomer unit to said cationic monomer unit comprising from about 55:45 to about 97:3; and
ii. a surfactant component;
iii. one or more cosmetic actives; and
iv. an aqueous carrier;
wherein upon dilution said personal care composition comprises one or more coacervates; and
b. subsequently removing said personal care composition off of said skin surface;
wherein the modification in the appearance of said skin surface comprises from about 1 to about 25% increase in a Delta L value as compared to a skin surface tropically treated with water.

19. The method of claim 18, wherein said modification in appearance comprises a opacity modification comprising a 1 to about 10% increase in Delta L measured in-vitro.

20. The method of claim 18, wherein said modification comprises a color modification comprising a 1 to about 10% increase in Delta L measured in-vitro.

21. The method of claim 18, wherein said modification comprises a gloss modification comprising a 1 to about 10% increase in Delta L measured in-vivo.
